(19) United States
(12) Patent Application Publication
(10) Pub. No.: US 2003/0125754 A1
(43) Pub. Date: Jul. 3, 2003

(54) Electrical hair buffing apparatus

(76) Inventors: Alice Davis, Ludington, MI (US); Nancy Zaino, Farmington, CT (US); Steven Wallace Ashworth, Richmond, VA (US); Kwok Kay Lee, Hong Kong (HK)

Correspondence Address: FRANCIS EDWARD MARINO, 394 MEREDITH NECK ROAD, MEREDITH, NH, 03253, US

(21) Appl. No.: 10/313,115

(22) Filed: Dec. 9, 2002

Related U.S. Application Data
(60) Provisional application No. 60/346,417, filed on Dec. 29, 2001.

(51) Int. Cl. A61B 17/50

(52) U.S. Cl. 606/133

(57) ABSTRACT

A hair-removing and exfoliating device is provided with a pair of counter-rotating abrasive surfaces for pressing against the skin to abrade hair there-from and to exfoliate the skin. The surfaces may be adapted for continual alternating movement in counter-rotating directions at the surface of the skin. In the preferred embodiment, two concentric counter-rotating abrasive surfaces are provided, one rotating clockwise and the other rotating counterclockwise.

### FIELD OF THE INVENTION:

This invention relates to a device for removing body and facial hair. More specifically, it relates to an electrical blade-less apparatus for removing hair by buffing and abrasion, while simultaneously exfoliating and providing beneficial effects to the skin.

### BACKGROUND AND OBJECTS OF THE INVENTION:

The most common solution to the problem of excess facial and body hair has been shaving. Numerous shaving devices have been marketed throughout the years that are intended to remove the hair by cutting it off near or below the surface of the skin. The main disadvantage of shaving is that to be effective, it must be done often. Blade shaving actually cuts and scraps and causes irritability and dryness to the skin. Shaving daily leaves stubble; hence it is not generally used by females in facial areas. Shaving is also blamed for causing various skin problems and irritations. The re-growth rate of cut hairs can be erratic and the skin can become more sensitive due to cuts and scraps, therefore causing inflammation.

Numerous epilation devices have been marketed that remove the hair by pulling it out. Similarly, hair has also been removed by applying mastic, which pulls out the hair when the mastic is removed. But hair-pulling is a painful process and is also blamed for increasing the rate of hair re-growth. Rather than pulling the hair root, it actually breaks off the hair shaft anywhere from high above or below the skin surface. Additionally, one must wait until the hair has grown to a sufficient length before it can be grasped for removal by mastic or pulling.

Electrolysis has been used as an alternative method for removing the hair by applying an electrical current to the hair follicle to kill the hair itself. This method is expensive and often requires professional assistance, or the utilization of a high cost appliance. And, each hair must be removed individually, rendering this approach tedious and time-consuming

Chemical means have also been used either to kill the unwanted hair or to bleach it to reduce visibility, but this has the disadvantage of causing peripheral damage to the skin.

Hand-held manual hair-sanding pads such as those disclosed in U.S. Pat. Nos. 1,910,647, 2,314,220 and 2,328,886 are pressed against the skin and rubbed to remove hair by sanding and abrasion. It is found however, that this technique is quite tedious and results are inconsistent, being dependent on uncontrollable qualities such as the pressure, speed, and frequency of the stroke applied by the user. Tiring and tedious arm motions are an inherent problem realized as the user performs the suggested manual (non-automated) sanding routine.

In U.S. Pat. No. 5,377,699 to Varnum, an electrical device is disclosed which is claimed to remove hair by abrasion. A rotating abrasive surface is pressed against the skin through a perforated web, which is claimed to wear the hair into a thinly tapered shape that is less visible. In practice, however, it is found that such abrasion that is continuously in the same direction is not effective in breaking the hair from and near the skin, as is most desirable. The distance between the blade and the skin, caused by the foil screen, is too great to achieve successful hair removal below the skin surface. Also, a damaged or broken foil could lead to cuts or nicks. In addition, replacement of the abrasive surfaces in the disclosed embodiments is quite burdensome, effectively restricting use of the device to one individual for sanitary reasons, or at the least, preventing timely replacement of the abrasive surface and reducing efficacy of the device.

U.S. Pat. No. 2,714,788 to Giovanna discloses an electrical apparatus for rotating an abrasive disk against skin to remove hair in which a mechanical mechanism causes the direction of rotation to alternate from clockwise to counter-clockwise after several rotations. There is a substantial amount of inertia developed by the rapidly rotating disk and associated components, which must be stopped instantly and redirected by this constant velocity mechanical design. Giovanna offers no means to decelerate the rotating head prior to reversal and to accelerate the head after reversal. As a result, Giovanna's apparatus is impractical for use by users who do not have extremely strong wrists. Such a mechanism is found to be quite prone to wear and tear and to cause user exhaustion.

In U.S. Pat. No. 5,084,046, an electrical apparatus is disclosed which includes a non-abrasive bearing surface on the face of a ring, having an eccentrically rotated abrasive disk there-within and coplanar there-with. The non-abrasive ring is held against the skin and the abrasive disk is rotated to remove hair from the skin within the ring. Because the disk is only adapted to rotate in one direction, this apparatus is found to be deficient in removing hair. Additionally, the skin area that is served by this device is small, so the removal of hair from a large area of skin is quite tedious.

It is therefore a primary object of the invention to provide a device that overcomes the deficiencies of the prior art and that effectively removes hair at or below the surface of the skin without pain or damage to the skin while simultaneously providing exfoliation of the skin.

It is a further object to provide such a device that removes hair by buffing and abrading it in a back and forth motion, or in a clockwise and counter-clockwise motion, to remove the hair most efficiently and completely.

It is a further object to provide such a device that operates consistently without tiring the user.

It is further object to provide such a device whose buffing and abrasive surface is easily replaced.

It is a further object to provide such a device that simultaneously provides beneficial effects to the skin while hair is being removed by removing dead skin cells and which helps to break down cellulite modules.

Further objects and advantages of the invention will become apparent upon review of the following description and depictions of various embodiments thereof.

### SUMMARY OF THE INVENTION

According to one embodiment of the invention, an electrical apparatus is provided that removes facial and body hair by buffing and abrading it repeatedly and alternately in a clockwise and counter-clockwise motion with a fine grit and non-allergenic buffing surface.

The apparatus is positioned with the buffing surface pressed against the skin to force the hair strands thereon to lie flat against the skin. While laying flat against the skin and continuously being spun three-hundred and sixty angular degrees in both directions, the hair shaft diameter is also being reduced. As a result of this positioning and the action of the buffing surface, the hair shafts are rotated in a continuous three-hundred and sixty angular degree motion. The abrasive surface is rotated across the skin, winding the long hair shafts into tightly wound knots. The abrasive surface is then rotated in the reverse direction, grasping the knots and twisting the strands in the opposite direction. Because the speed of rotation is controlled electronically, the rotation can be decelerated just prior to reversal and accelerated back to the desired rotational speed after reversal to reduce the discomforting effects to the user and reduce wear and tear on the motor.

Repeated and rapid reversals of the rotation of the buffing and abrasive surface causes flexure of the hair strands near to their roots and below the surface of the skin, while simultaneously reducing the thickness of the hair shaft. This repeated flexure eventually causes the hair shafts to become weakened and break off or to be buffed to a fine point at the point of flexure, always at or below the surface of the skin. Visibility is reduced, and the hair root is left intact with this repeated process. The removal of the hair shaft to below the skin surface, by exfoliating the skin, additionally results in a smoother skin surface.

It is also found that buffing has a beneficial effect on the skin itself, removing dead skin cells, smoothing roughness of the skin and reducing cosmetic flaws, such as rosatia and cellulite, by continuous stimulation. The skin surface will therefore respond to this vigorous agitation in a positive and healthy manner. After continuous buffing and exfoliating, hair growth becomes less vigorous. Buffing actually removes tiny hair shafts at the root level, consequently causing no future growth of that hair follicle. By comparison, traditional blade shaving provides little or no stimulation and actually damages the skin.

According to the preferred embodiment of the invention, an electrical apparatus is provided with a planar buffing disk that rotates continuously in one direction. The disk is surrounded concentrically by a planar buffing ring, which rotates in the opposite direction. The movement of the apparatus back and forth across the surface of the skin while pressing the counter-rotating buffing surfaces against hair on the skin causes a similar knotting and abrading effect that ultimately removes the hair by breaking it off at or below the surface of the skin. The hair is removed most efficiently where the inside of the ring meets the outside of the disk. The hair shafts are being spun in both directions at eight-hundred revolutions per minute, with a slight hand movement of the apparatus on the skin surface.

Another embodiment of the invention includes two or more co-planar side-by-side rotating or counter-rotating buffing disks. It is also found that either of an orbital or a random motion of the buffing surface, or a linear back and forth motion, coupled with the application of pressure to the hair by the buffing surface, provides similar, although not optimal results. Various other embodiments are also anticipated and well within the scope of the invention, which is most simply described as the application of a back and forth buffing action against the skin with an abrasive surface, by an electrically energized apparatus.

The invention will be better understood and additional objects and advantages will become apparent from the following descriptions taken in connection with the accompanying drawings.

### BRIEF DESCRIPTION OF DRAWINGS

FIG. 1 is a side view of a hair-buffing apparatus in accordance with the preferred embodiment of the invention, having a portion of the housing thereof broken away to show the planetary gearing within,

FIG. 2 is a bottom perspective view of the apparatus of FIG. 1,

FIG. 3 is a bottom plan view of the apparatus of FIG. 1,

FIG. 4 is an enlarged side view of the inner workings of the apparatus of FIG. 1,

FIG. 5A is a side view showing the interfacing of the apparatus of FIG. 1 with skin and hair,

FIG. 5B is an enlarged partial side view of the interface of the apparatus of FIG. 1 with the skin and hair during buffing,

FIG. 5C is a progressive series of images depicting a hair after various amounts of buffing,

FIG. 6 is an exploded perspective view of the apparatus of FIG. 1,

FIG. 7 is an electrical schematic diagram of the apparatus of FIG. 1,

FIG. 8 is a side view of a hair-buffing apparatus according to a second embodiment of the invention,

FIG. 9 is a bottom perspective view of the apparatus of FIG. 8,

FIG. 10 is a bottom plan view of the apparatus of FIG. 8,

FIG. 11 is a bottom plan view of a hair-buffing apparatus according to a third embodiment of the invention,

FIG. 12 is a bottom plan view of a hair-buffing apparatus according to a fourth embodiment of the invention,

FIG. 13 is a bottom plan view of a hair-buffing apparatus according to a fifth embodiment of the invention,

FIG. 14 is a partial perspective view of an alternate gear system for use in an embodiment such as the first embodiment,

FIG. 15A is a partial cross-sectional view through the gear system of the apparatus of FIG. 1,

FIG. 15B is a partial side view of the gear system of the apparatus of FIG. 1,

FIG. 15C is a detailed partial cross-sectional view through the gear system and motor of the apparatus of FIG. 1,

FIG. 15D is a detailed partial top perspective view of the gear system and motor of FIG. 14,

FIG. 15E is a detailed partial side view of the gear system and motor of FIG. 14,

FIG. 16 is a top perspective view of the apparatus of FIG. 1 showing the abrasive elements and associated components exploded there-from,

FIG. 17A is a cross-sectional side view of the apparatus of FIG. 1 showing the abrasive elements and associated components exploded there-from,

FIG. 17B is a cross-sectional side view of the apparatus of FIG. 1 showing the abrasive elements and some associated components exploded there-from,

FIG. 18A is a full side view of the apparatus of FIG. 1 showing the abrasive elements and associated components exploded there-from,

FIG. 18B is full side view of the apparatus of FIG. 1 showing the abrasive elements exploded there-from,

FIG. 19A is a bottom perspective view of the apparatus of FIG. 1 showing the abrasive elements and their associated components exploded there-from,

FIG. 19B is a bottom perspective view of the apparatus of FIG. 1 showing the abrasive elements exploded there-from,

FIG. 19C is a top perspective view of the apparatus of FIG. 1 showing the abrasive elements and some associated components exploded there-from,

FIG. 19D is a bottom perspective view of the apparatus of FIG. 1 showing the abrasive elements and some associated components exploded there-from,

FIG. 20A is a partial cross-sectional side view through the abrasive elements and associated components of the apparatus of FIG. 1,

FIG. 20B is a partial top perspective view of the abrasive elements and associated components of the apparatus of FIG. 1,

FIG. 21 is a partial top perspective view of the abrasive elements and some associated components of the apparatus of FIG. 1,

FIG. 22 is a bottom perspective view of the drive connector subassembly of the apparatus of FIG. 1,

FIG. 23A is a partial exploded bottom perspective view of the abrasive pad connector subassembly of the apparatus of FIG. 1,

FIG. 23B is a partial bottom perspective view of the abrasive pad connector subassembly of the apparatus of FIG. 1,

FIG. 23C is a partial top perspective view of the abrasive pad connector subassembly of the apparatus of FIG. 1,

FIG. 24 is a top perspective view of the ring pad connector of the apparatus of FIG. 1,

FIG. 25 is a top perspective view of the disk pad connector of the apparatus of FIG. 1,

FIG. 26 is a bottom perspective view of the outer drive connector of the apparatus of FIG. 1,

FIG. 27 is a bottom perspective view of the inner drive connector of the apparatus of FIG. 1,

FIG. 28A is a partial exploded bottom perspective view of the abrasive pad connector/drive connector subassembly of the apparatus of FIG. 1,

FIG. 28B is a partial bottom perspective view of the abrasive pad connector/drive connector subassembly of the apparatus of FIG. 1,

FIG. 28C is a partial bottom perspective view of the abrasive pad connector/drive connector subassembly of the apparatus of FIG. 1,

FIG. 29 is an enlarged partial perspective view of the interface between the disk pad connector and the inner drive connector of the apparatus of FIG. 1,

FIG. 30 is an enlarged partial perspective view of the interface between the disk pad connector and the ring pad connector of the apparatus of FIG. 1,

FIG. 31 is a partial top perspective view of the motor and abrasive elements of a seventh embodiment of the invention,

FIG. 32 is a partial top perspective view of the motor and abrasive elements of an eighth embodiment of the invention, and

FIG. 33 is a partial top perspective view of the motor and abrasive elements of a ninth embodiment of the invention,

### DETAILED DESCRIPTION OF THE PREFERRED EMBODIMENT

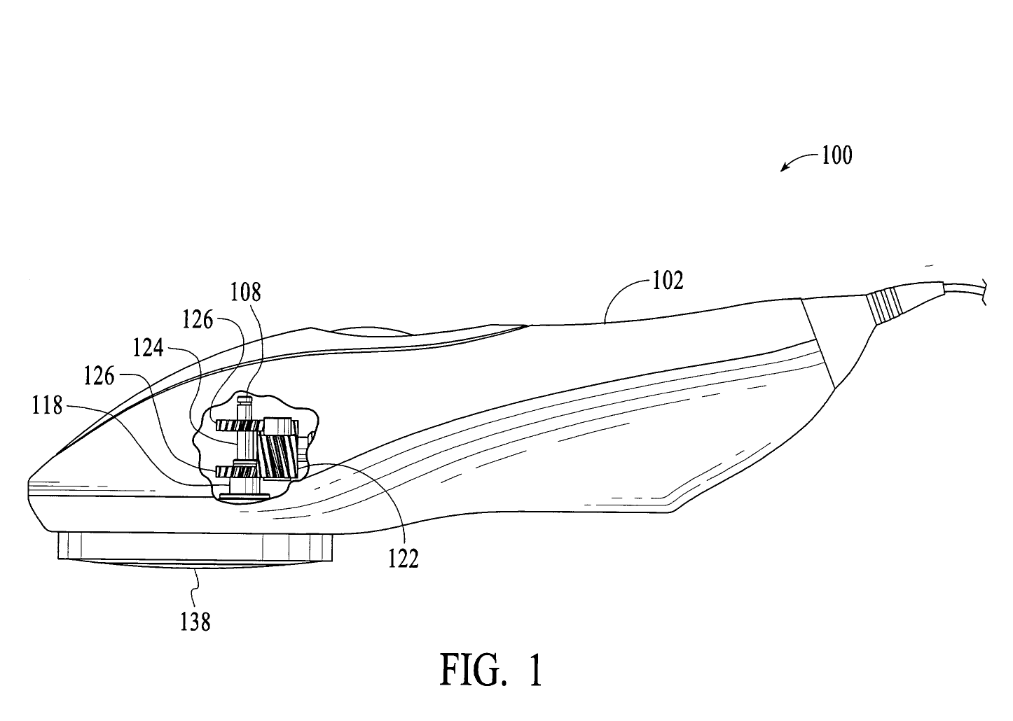

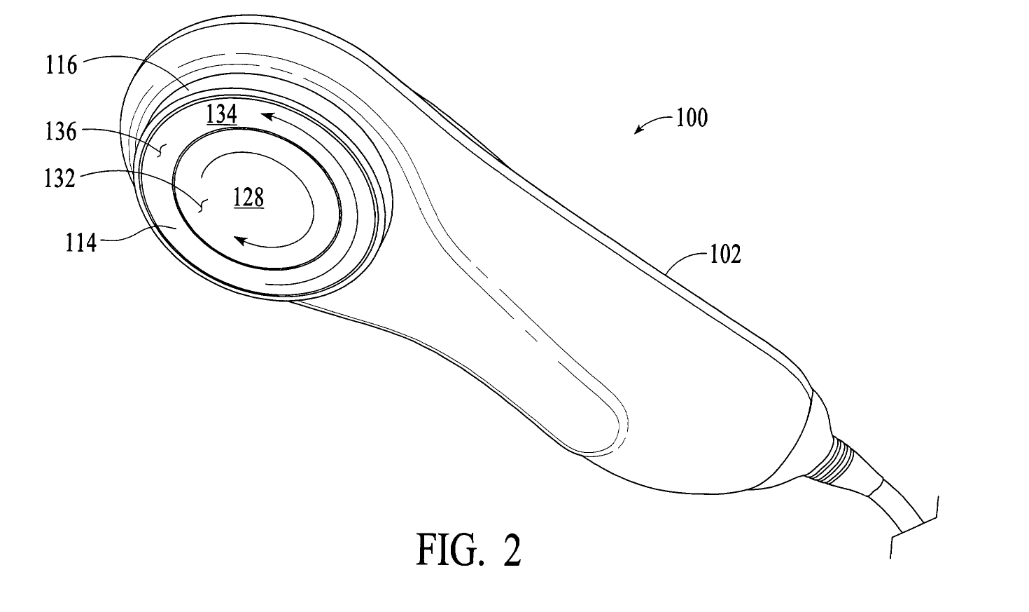

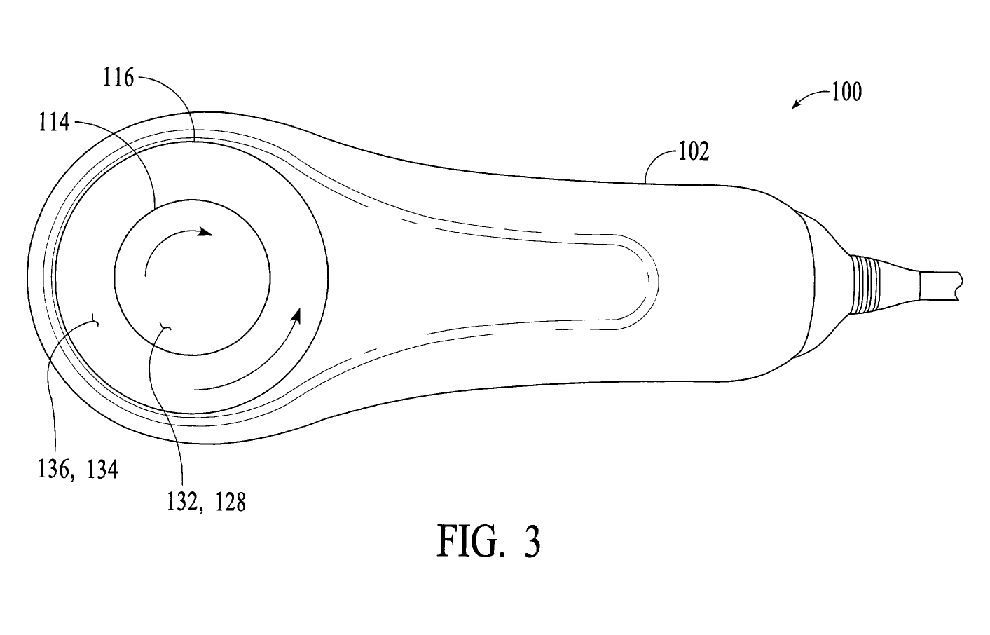

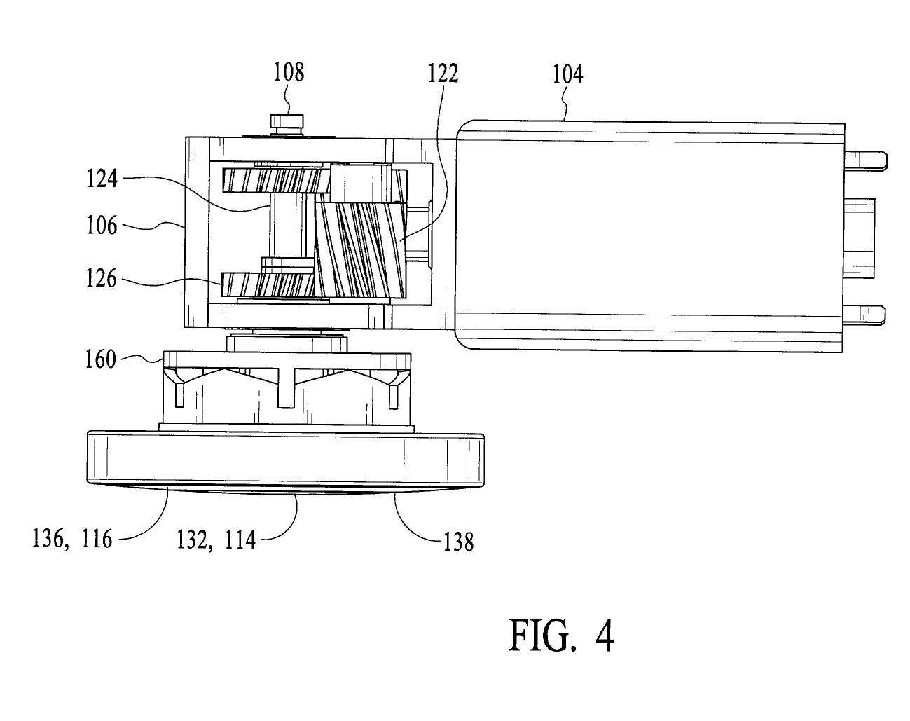

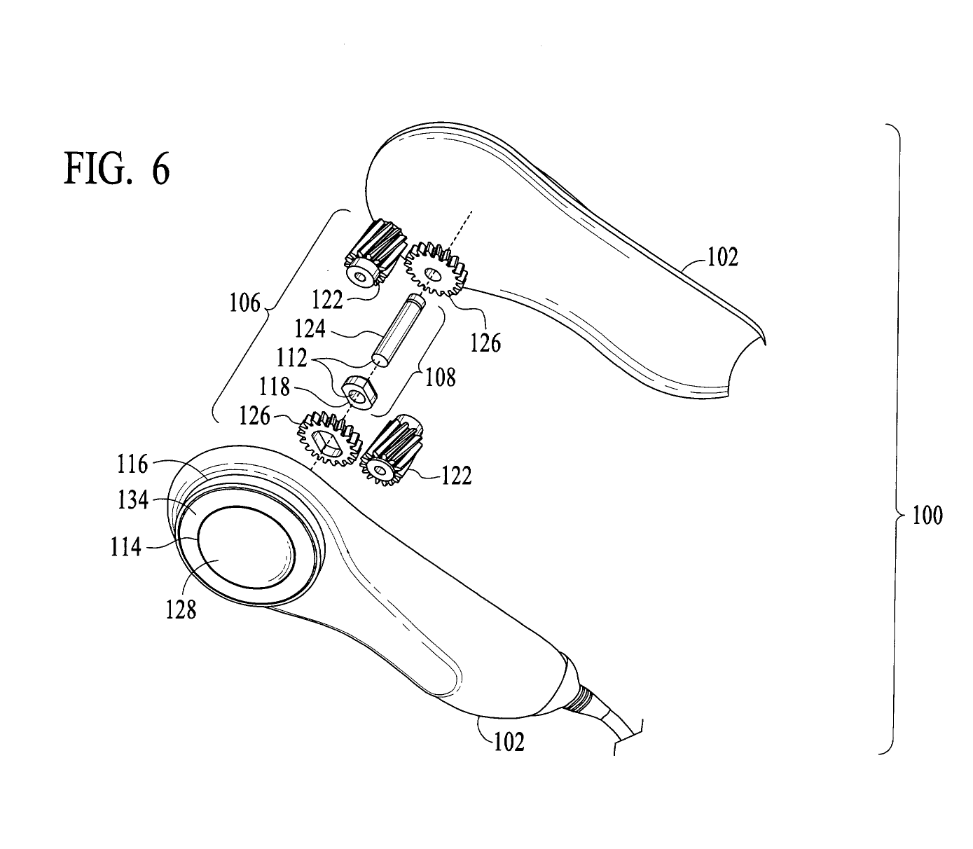

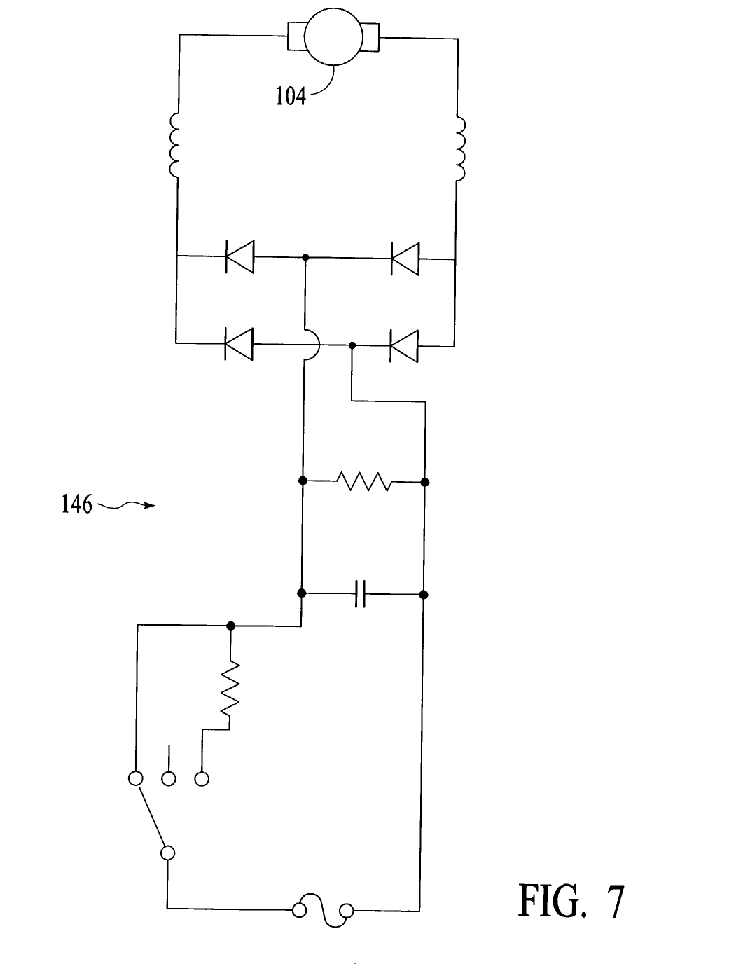

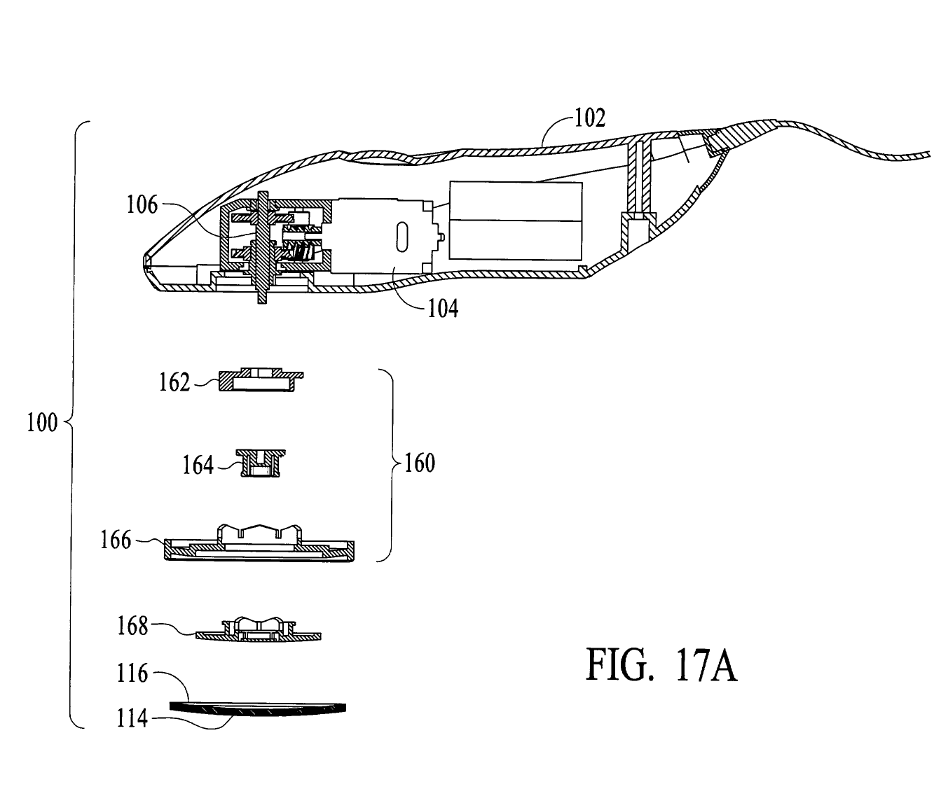

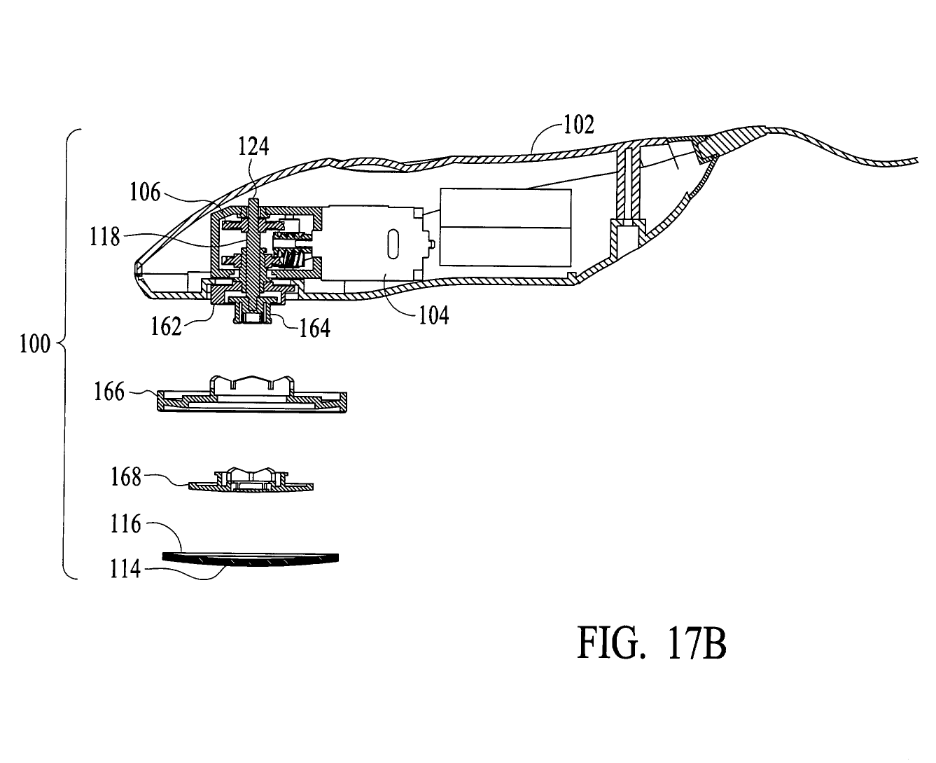

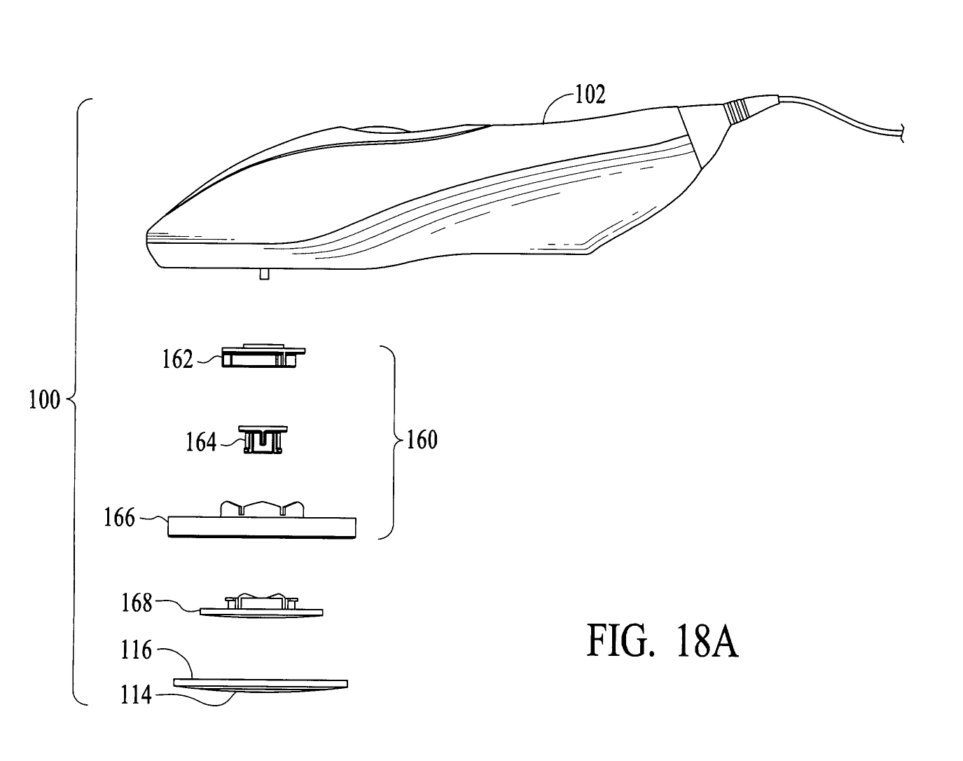

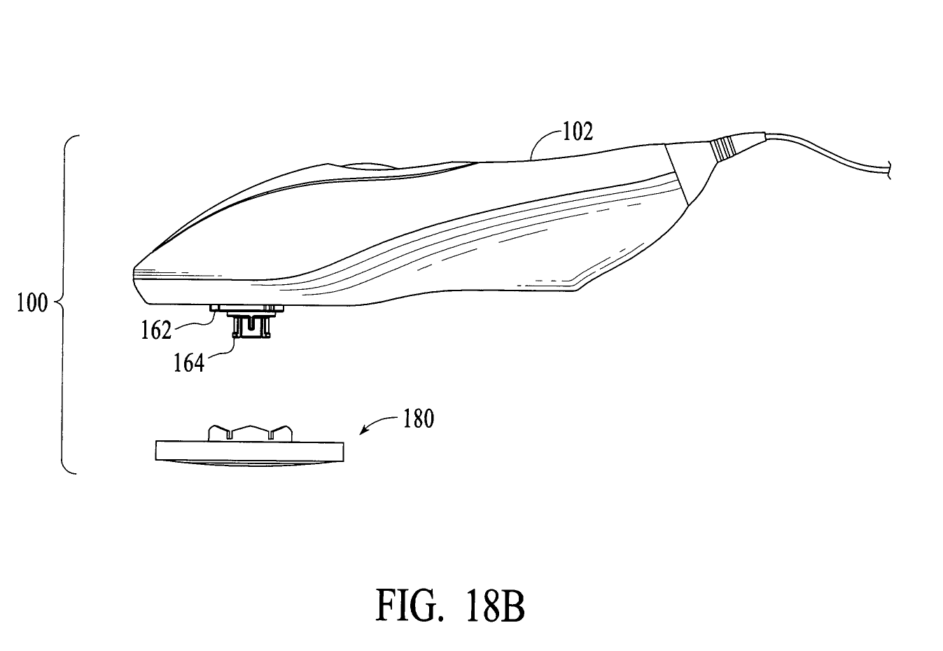

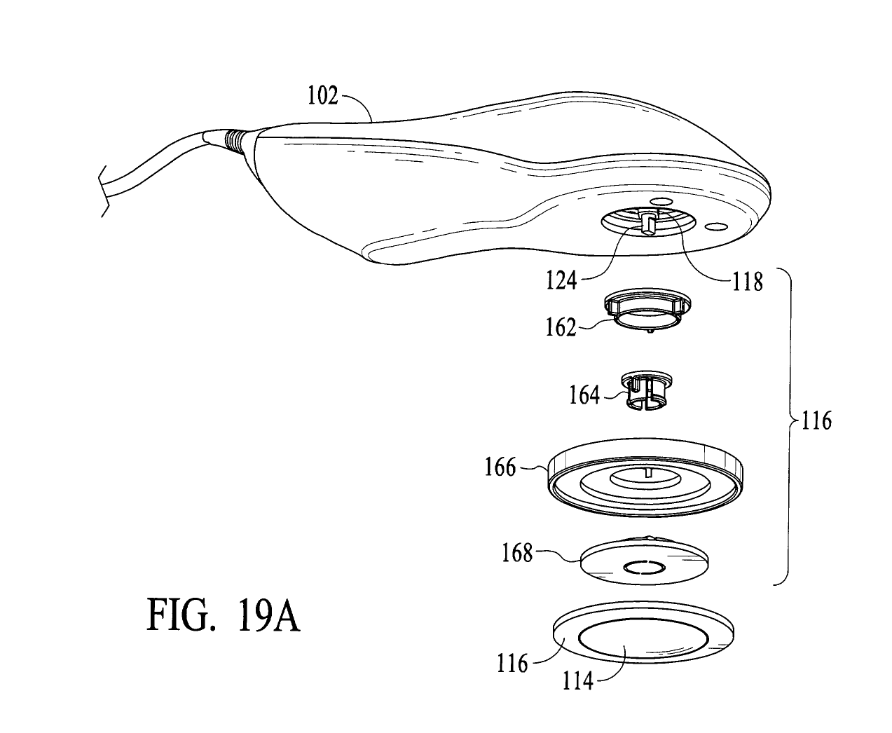

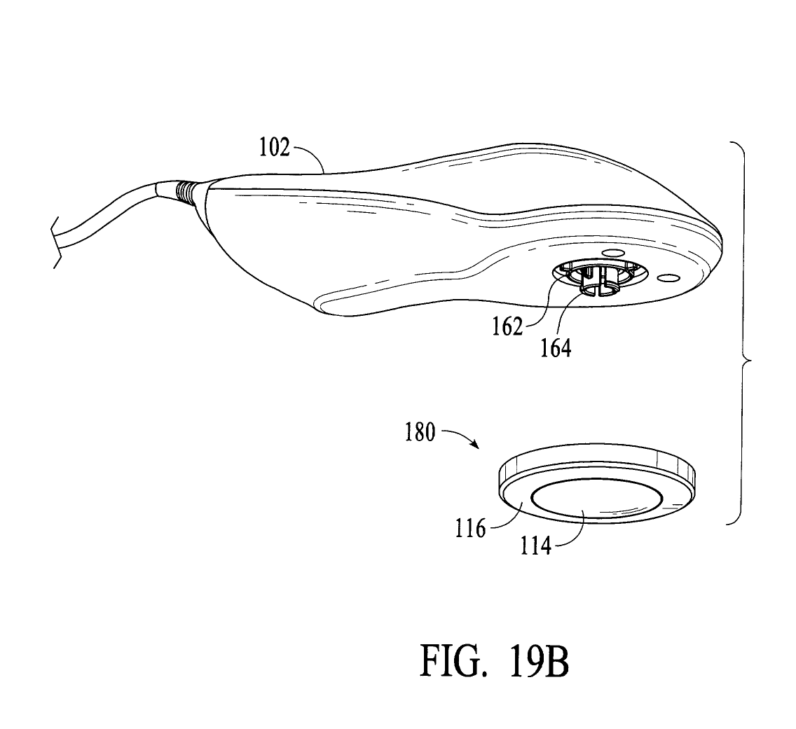

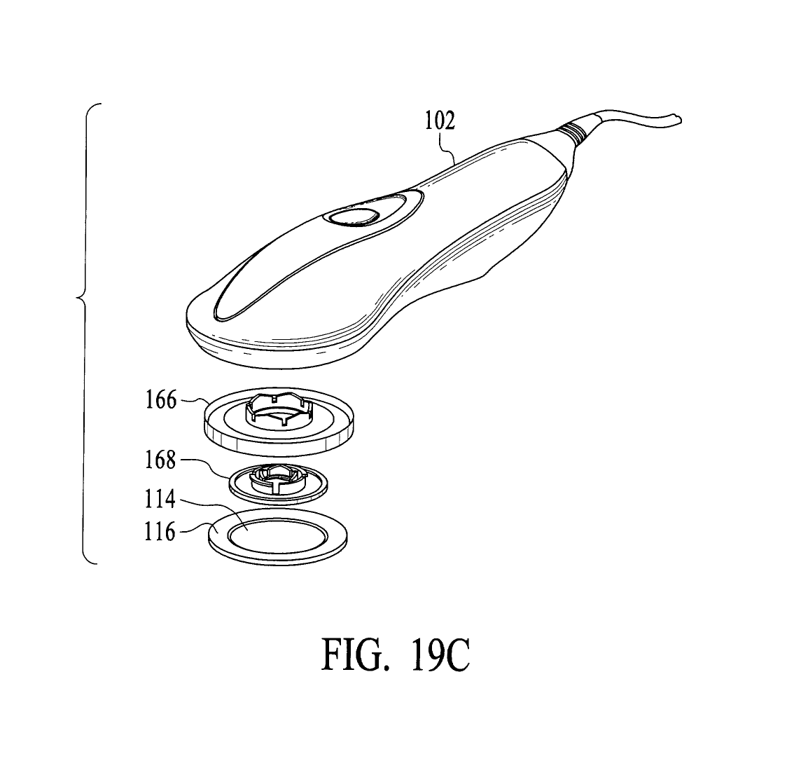

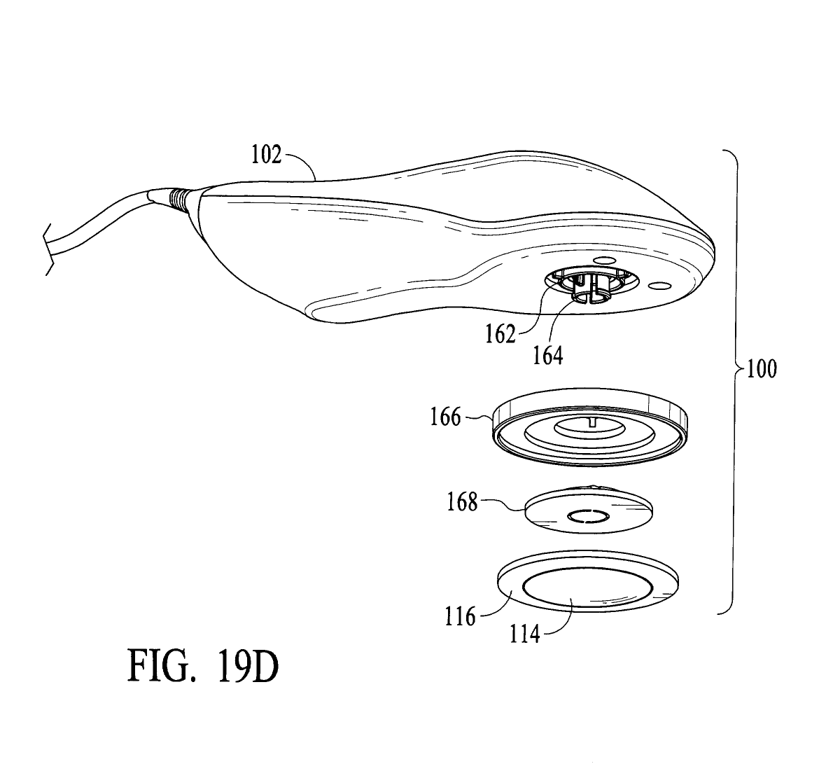

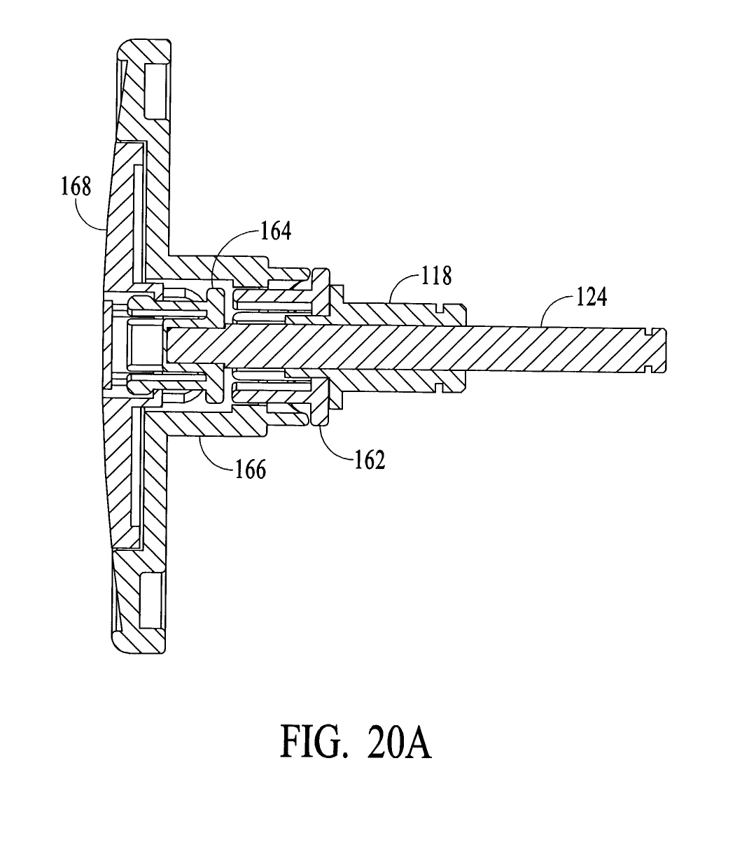

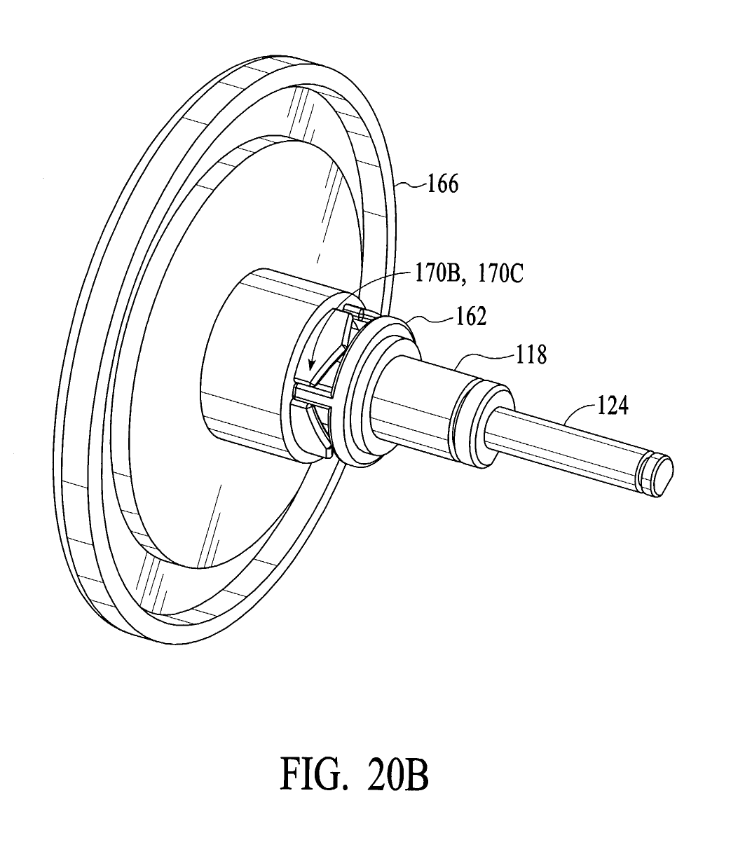

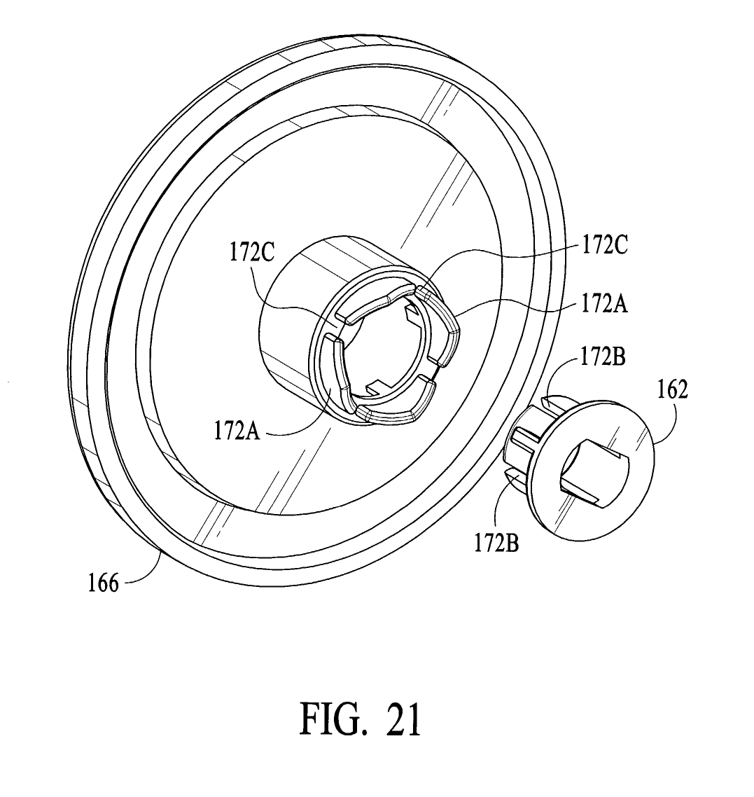

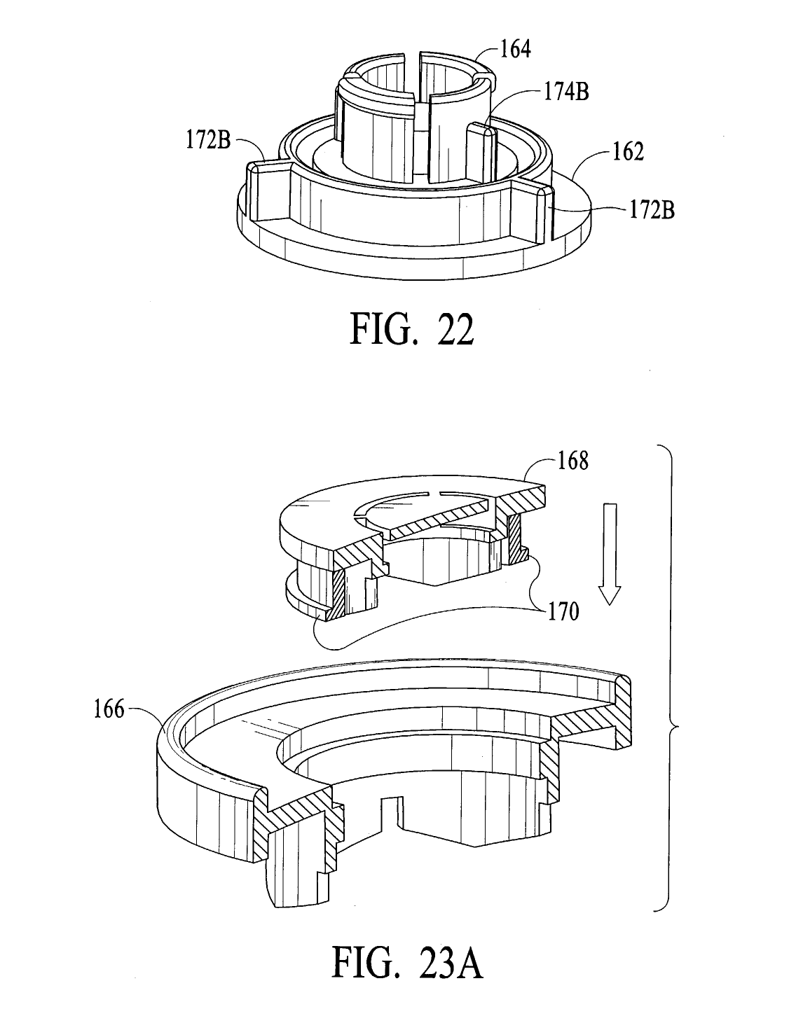

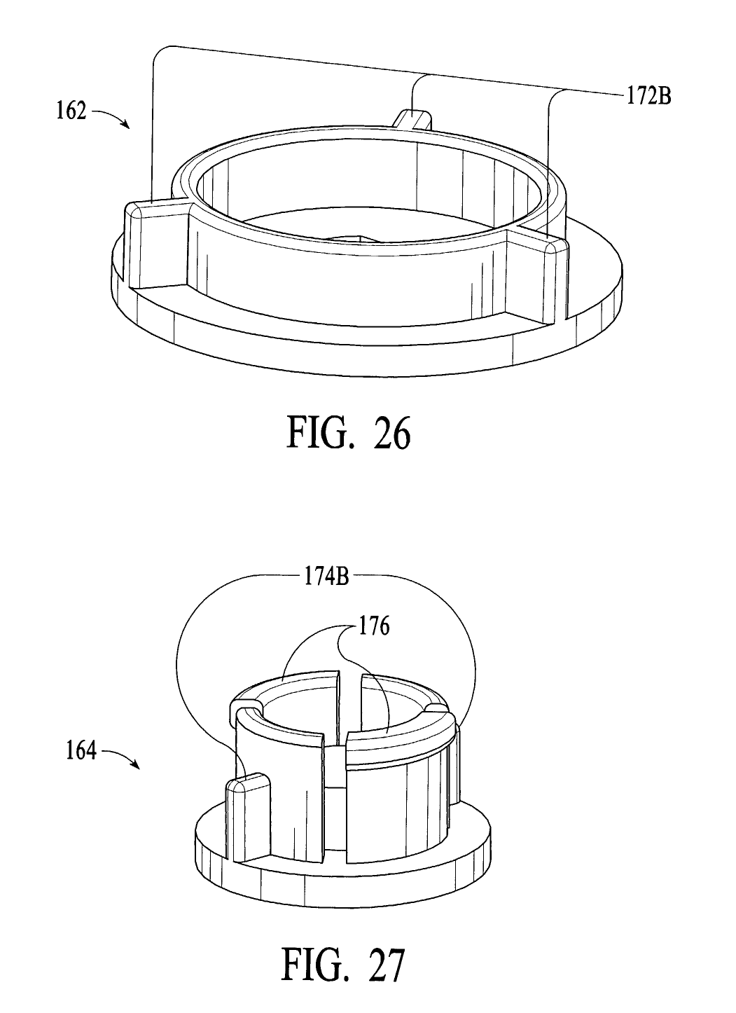

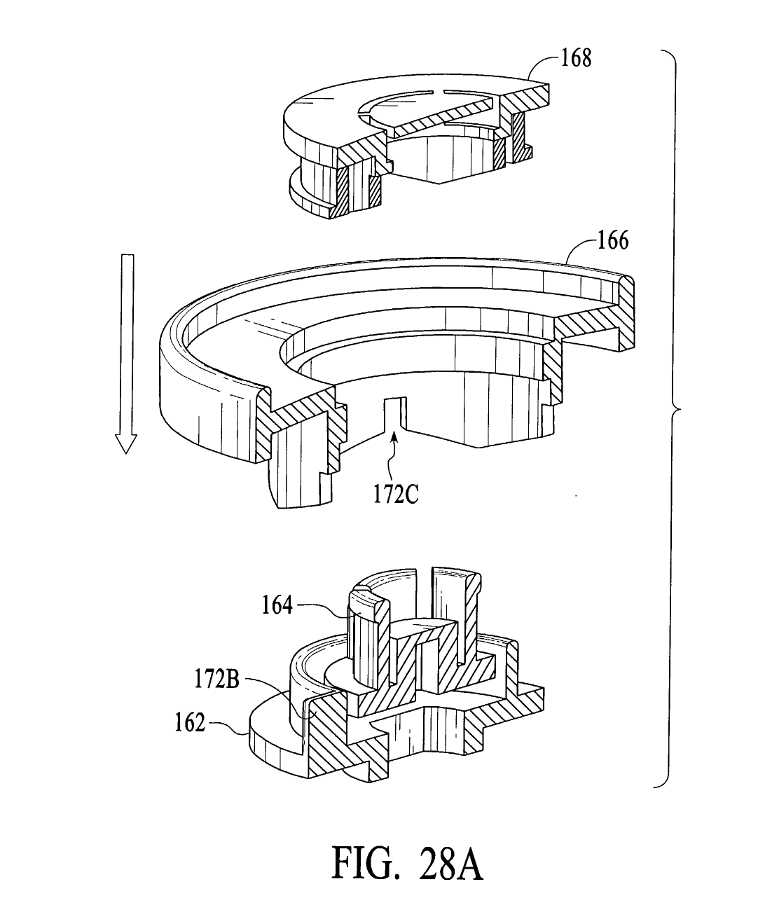

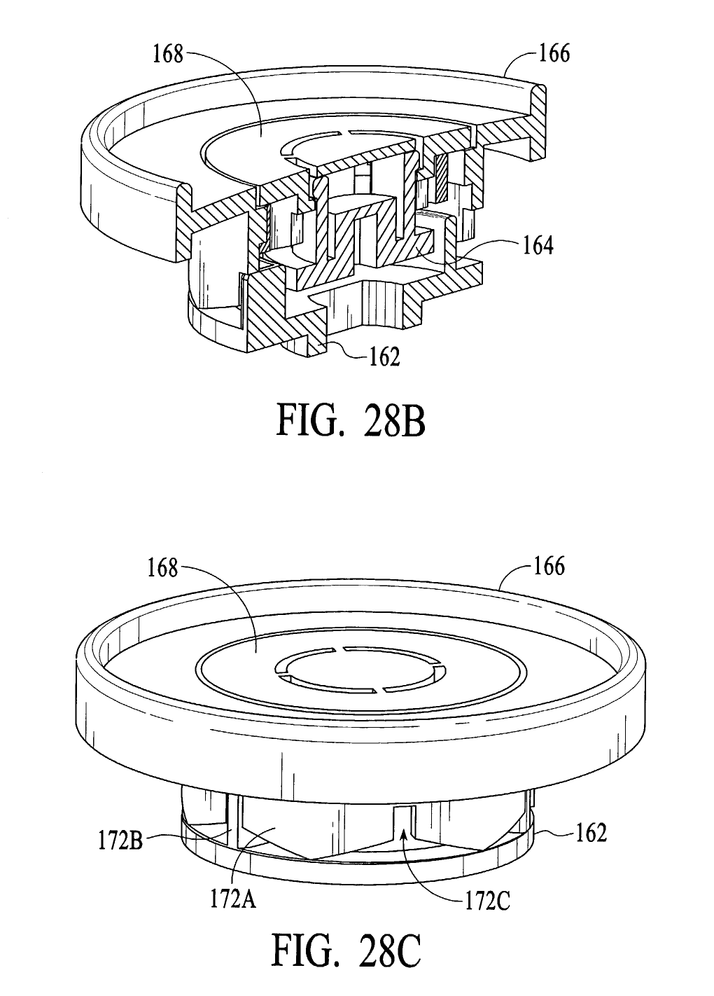

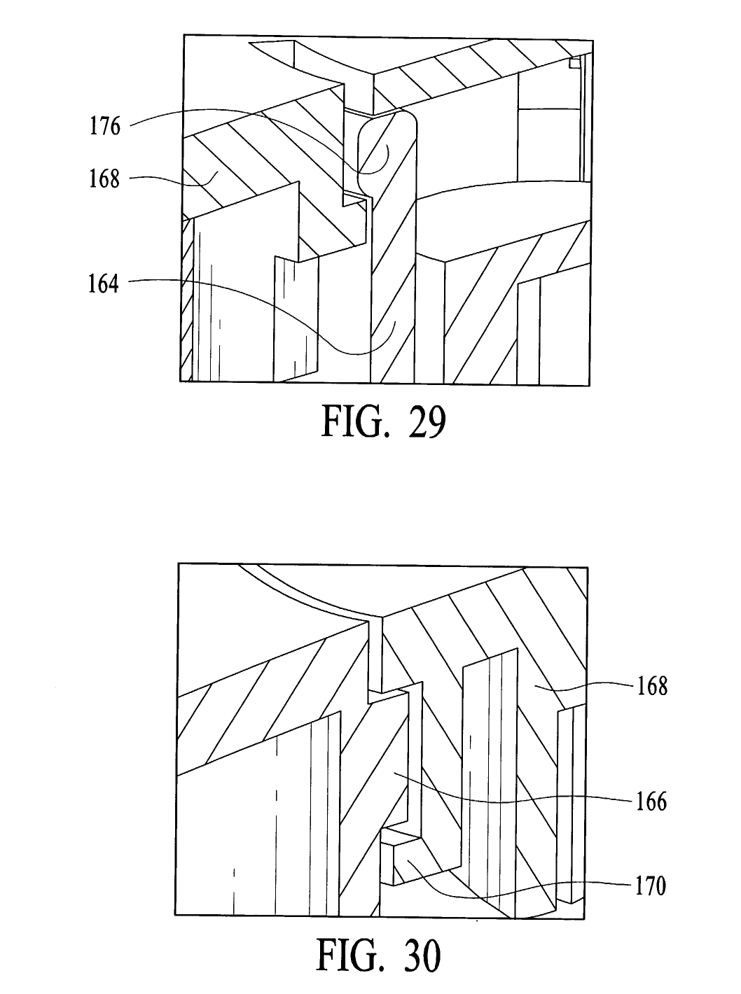

Various abrading apparatuses in accordance the invention are shown in the accompanying FIGS. Referring first to FIGS. 1 through 7 and FIGS. 16 through 30, an electrical apparatus 100 according to the preferred embodiment of the invention is shown which effectively abrades hair from skin.

Housing 102 includes within an electric motor 104 and a planetary gear system 106 in communication therewith. From the gear system and the housing extends a coaxial shaft assembly 108 having a coupling feature at its terminal end 112 for releasably accepting and accommodating a buffing disk 114 and a circumferential buffing ring 116. The shaft assembly comprises hollow outer shaft 118 communicating with the gear system's “planet” gears 122 and comprises inner shaft 124, coaxially displaced within the hollow shaft and communicating with the gear system's “sun” gear 126. Rotation of motor 104 causes transmission through the gear system 106 to coaxially rotate disk 114 and ring 116 in opposite directions.

A first abrasive surface 128 is comprised on the spherical face 132 of the buffing disk 114 and a second abrasive surface 134 is comprised on the frusto-spherical face 136 of the buffing ring 116. It can thus be appreciated that energization of motor 104 causes the abrasive surfaces, 128 and 134, to rotate about the axis of shaft assembly 108, with the first and second abrasive surfaces rotating in opposite directions.

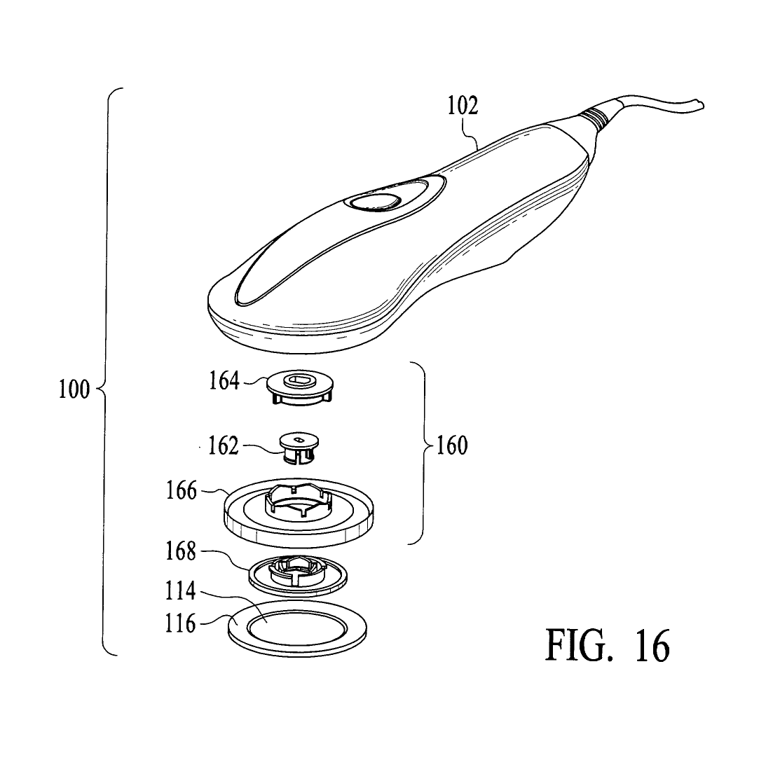

The disk 114 and ring 116 are individually removable from the terminal end 112 of the shaft assembly 108 by means of the connector assembly 160 depicted in FIGS. 16 through 30. But it is also anticipated that various other attachment means could be employed to removably affix the abrasive surfaces, 128 and 134, to the apparatus 100, so that they may be removed occasionally for cleaning or replacement. Such other attachment means may include threading, adhesive-backing, or may even simply be in the form of hook and loop fastening between the abrasive surfaces, 128 and 134, and the flat faces, 132 and 136.

Each abrasive surface is comprised of a very finely gritted sandpaper or emery cloth. The disk 114 and ring 116 are shaped and arranged so that faces 132 and 136 lie on the same spherical surface to form a single buffing surface 138 which is intended to be pressed against the hairy skin 142 in use. Alternatively, the faces of the disk and ring could be flat and co-planar.

Connector assembly 160 comprises outer drive connector 162, inner drive connector 164, ring pad connector 166 and disk pad connector 168. Outer drive connector 162 is permanently attached to outer shaft 118 and inner drive connector 164 is permanently attached to inner shaft 124. In this preferred embodiment, disk 114 and ring 116 are permanently attached to disk pad connector 168 and ring pad connector 166 respectively, for replacement together. However, it is also anticipated that the disk and pad could be replaced independently of their pad connectors and affixed thereto by such means as “peel-and-stick” adhesive backing or hook and loop fastening.

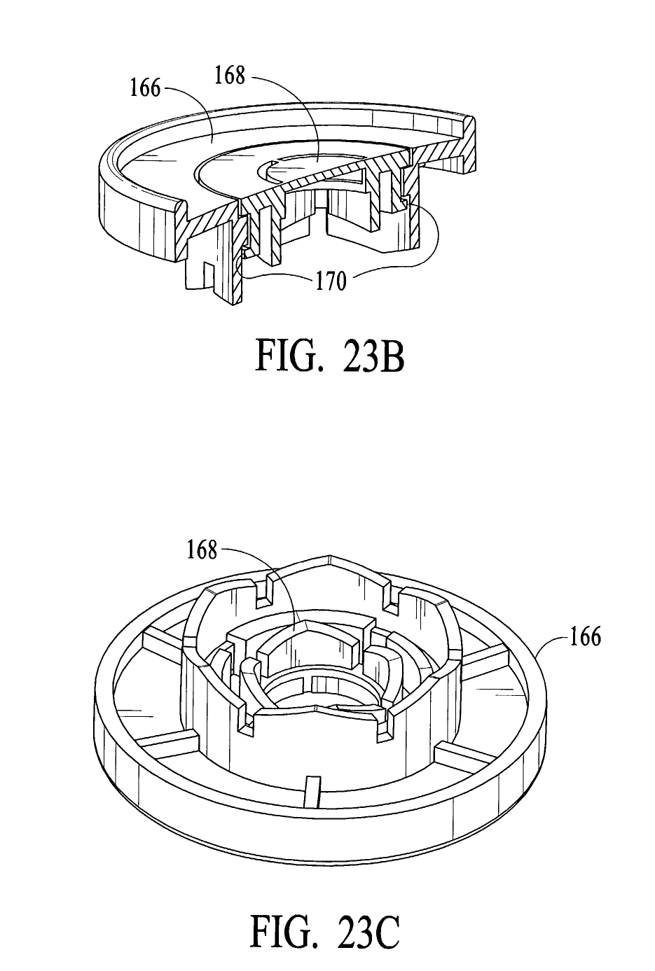

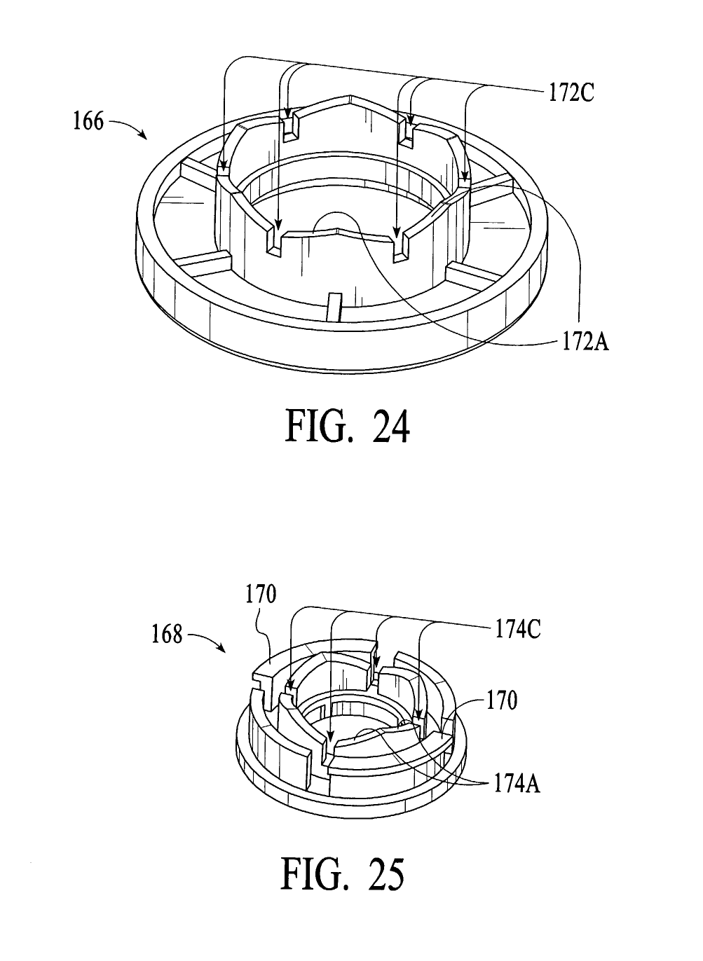

As preferred, removal and replacement of the disk 114/disk pad connector 168 assembly and of the ring 116/ring pad connector 166 assembly is effected by a snap fitting between the pad connectors and their associated inner drive and outer drive connectors, 164 and 162. Initially, the disk pad connector 168 is snapped into the ring pad connector 166 as shown in FIGS. 23A though 23B, so that the two components are fixed longitudinally by disk pad locking features 170, but free to rotate relative to each other. As this ring/disk pad connector subassembly 180 is pushed axially onto the inner and outer drive connectors, 164 and 162, lead-in features 172A of the ring pad connector and 174A of the disk pad connector are engaged by tabs 172B of the outer drive connector and 174B of the inner drive connector, respectively, to cause alignment such that tabs 172B and 174B find and enter slots 172C and 174C, respectively, and the ring and disk pad connector thus become rotationally fixed to the outer and inner drive shafts, 118 and 124, respectively. As best seen in FIGS. 24 and 25, lead-in features 172A and 174A are tapered inwardly toward slots 172C and 174C respectively, to cam the tabs into the slots, so that any initial misalignment of the tabs and slots will be corrected by the combination of the axial attaching force and the camming action of the lead-ins. Locking features 176 of the inner drive connector grasp the disk pad connector to affix the subassembly 180 axially and thus to hold the ring 116 and disk 114 pads onto the device housing 102. Rotation of motor 104 thus enables rotation of the gear system 106, enabling countering rotations of the inner 124 and outer 118 shafts, and of the abrasive ring 116 and disk 114 thereby.

In use, the buffing surface 138 is pressed against the skin 142 containing the hair 144 to be removed and the motor 104 is energized. Circuitry 146 is employed to control the motor speed so that the disk 114 and ring 116 both rotate at approximately 800 RPM during normal rotation. The circuitry is also adjustable by the user to cause faster and slower rotation speeds. While rotation is enabled and the buffing surface 138 is being pressed against the hairy skin 142, the apparatus 100 is moved back and forth on the skin to accentuate the effects of the counter-rotating abrasive surfaces, 128 and 134, and to repeatedly and alternately expose all hair strands 144 on the skin 142 to both rotating surfaces. This process is continued as the hair is knotted, flexed, abraded and broken off at or just below the surface of the skin. Eventually, every hair strand is removed. It is found that most hair removal action occurs at the junction or separation of the disc and ring, at the location where they are moving simultaneously in opposite directions.

Not only is this counter-rotating arrangement found to increase motor life by eliminating rotational inertia and torque variations compared to alternative embodiments of the invention, but also balance of the whole device is improved and user fatigue is found to be reduced.

As shown, the gearing 106 is arranged to provide equal rotational speed to both of the inner shaft 124 and outer shaft 118 and so to both abrasive surfaces. However, it is anticipated that the selection of alternate gear ratios can modify the relative speeds of rotation of the shafts.

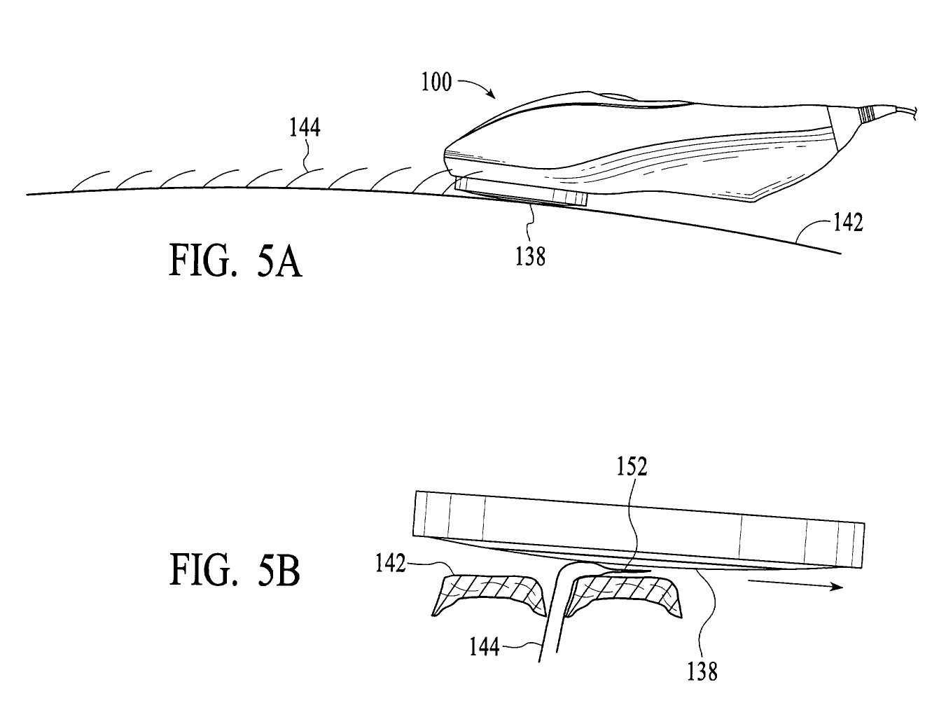

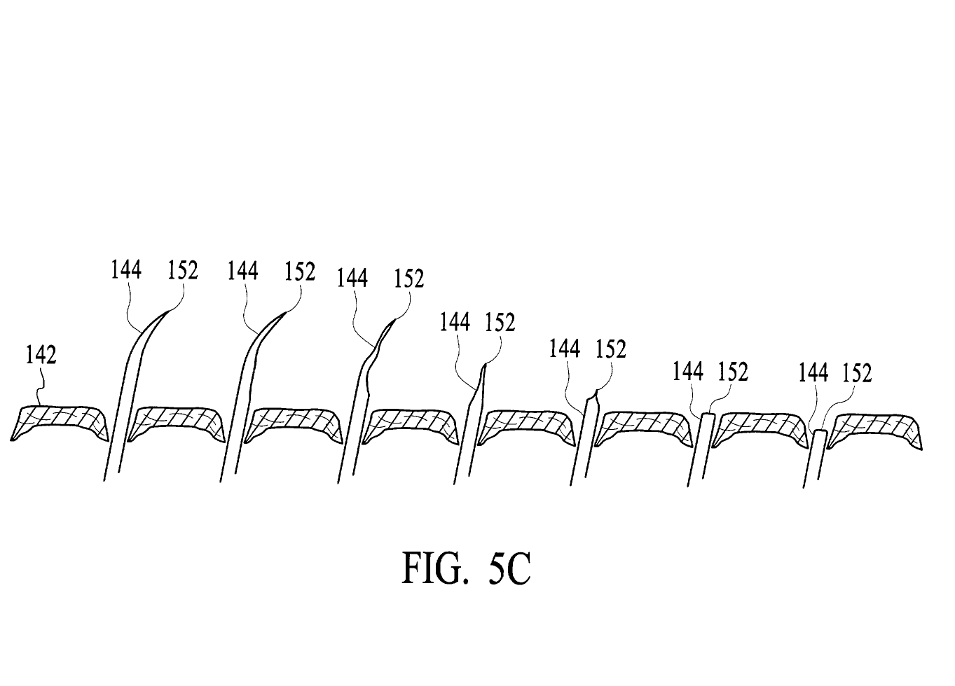

FIG. 5C depicts a progressive series of views of a long hair shaft 144 as it is buffed by the present invention. Viewed from left to right, the hair shaft first stands straight up in the vertical position prior to buffing and then is forced to lie in a horizontal position against the skin 142 by the pressing of the apparatus against the skin, as best seen in FIG. 5C. As buffing progresses, the hair shape is transformed as viewed by moving rightwardly in FIG. 5. It can be seen that the hair shaft is sharpened at its tip 152. It is found that the hair shaft is actually wound and buffed clockwise and counter-clockwise very rapidly as the apparatus is passed back and forth across the skin to cause the hair shaft to knot and break near to the skin. Buffing continues as the hair is reduced to just below the skin as shown to the right. Additional buffing then exfoliates the skin to remove dead cells.

For shorter hair shafts, knotting rarely occurs, but the buffing action reduces the thickness of the hair until to breaks of at the skin surface, then additional buffing reduces the hair to just below the skin surface and exfoliates the skin.

For very fine and lightly rooted hairs, such as new hairs, it is found the abrasive action actually pulls the entire hair shaft out by the root, without pain or discomfort. It has been stated in journals that blade shaving of lightly-rooted (new) hairs will actually promote subsequent aggressive hair growth.

The efficiency of this hair removal is greatly enhanced by keeping constant pressure against the skin. If the unit is removed from the skin surface before the hair shaft is taken below the epidermis, then the process will take longer because the longer hair shafts may become unknotted and must be knotted again.

The hair shaft is reduced to below the skin because of the pressure being exerted onto the skin precisely where the abrading surface is contacting the hair. This pressure compresses the skin in the area surrounding the hair shaft to expose more of the shaft during removal. This is contrary to a blade-shaving device where exerting added pressure on the skin surface will cause detrimental scraping and or cutting of the skin. Also, electric shavers cannot cut the hair to below the skin because they include screens between the cutters and the skin, which prohibit similar compression of just the skin.

An additional benefit of the present invention is its ability to exfoliate the epidermis by abrading dead cells from the skin. During the buffing routine, the user will notice a white power substance forming around the buffing site. This is evidence of the exfoliation taking place—the actual removal of dead skin cells, minor skin and hair bumps, and lumps and exposed cellulite. Once the hair and the dead epidermis are removed, the skin surface is very soft and smooth.

Other benefits to the skin and reduction in toil are realized by not having to use water, shaving cream and soaps (which are natural skin drying agents). Also, the buffing paper employed is very fine grit on a non-allergenic paper to avoid skin reactions.

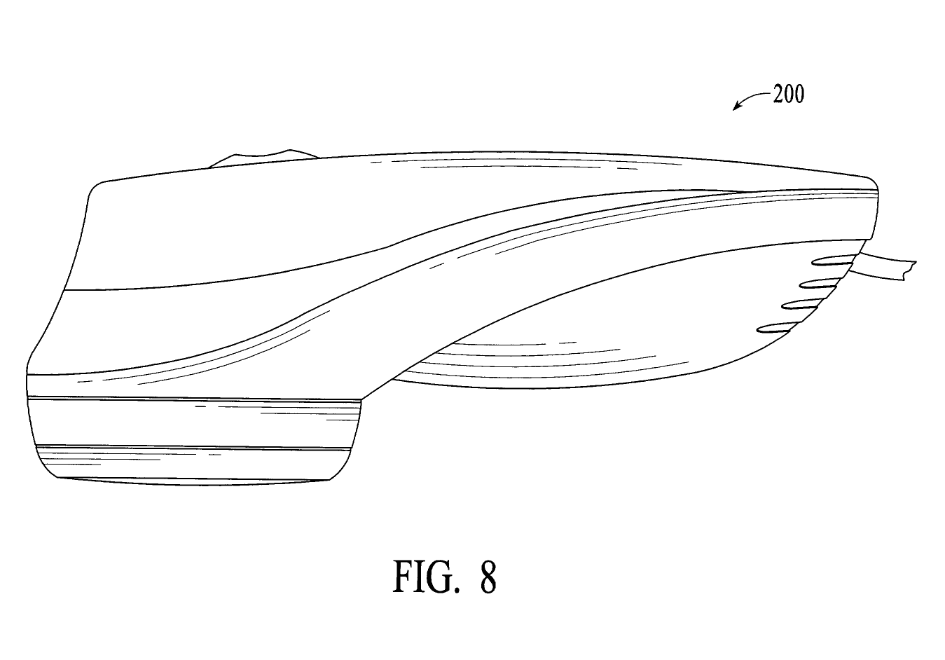

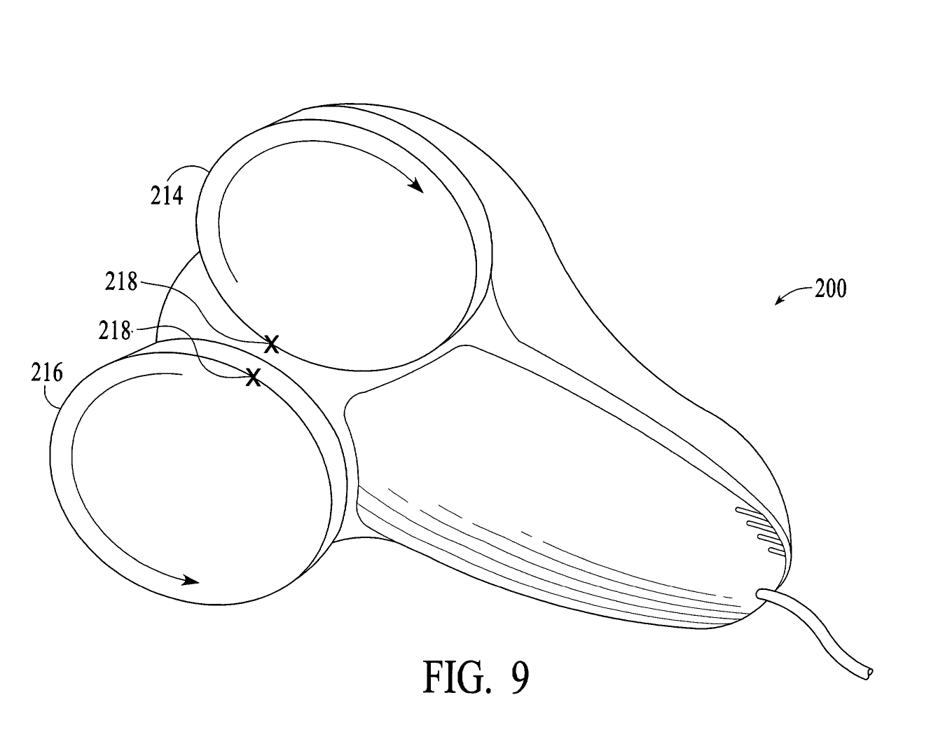

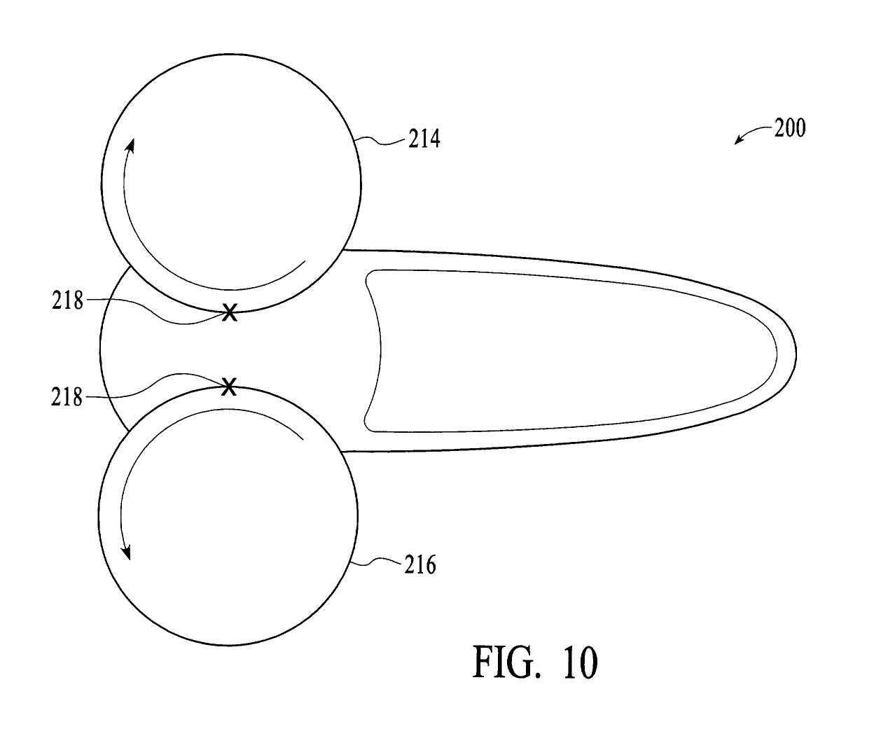

A second embodiment of the invention 200 is depicted in FIGS. 8 though 10 and includes two side-by-side disks, 214 and 216. Preferably, these disks are arranged to rotate in opposite directions, as shown. This causes similar knotting and reverse-knotting of the hairs as the apparatus is passed back and forth over the hairy skin. It is anticipated however, that the disks could rotate in the same direction, thereby causing the abrasive surfaces in their points of tangency 218 to move oppositely and act on the hair in that area similarly to the preferred embodiment 100.

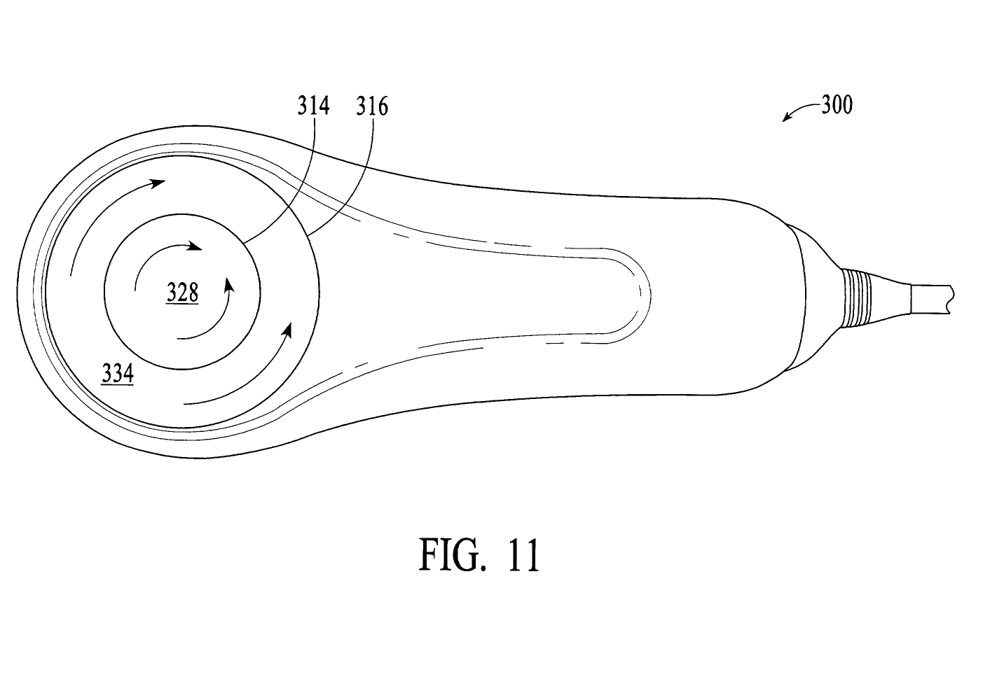

A third embodiment 300 is depicted in FIG. 11. This apparatus is constructed similarly to the preferred embodiment 100, except that the circuitry causes the motor rotation to reverse repeatedly and rapidly during use. Connected to the motor's rotation through similar gearing, the disk 314 initially rotates on one direction, say counter-clockwise, and the ring 316 rotates in the other direction, say clockwise, then after 1 to 3 complete rotations, the motor direction, and thus the disk's and ring's rotation directions, are reversed, so that the disk rotates clockwise and the ring rotates counter-clockwise. Again, after 1 to three complete rotations in these directions, the motor direction is electronically slowed to a stop, then reversed so that the disk and ring rotations reverse again. The frequency of rotation reversals should occur in the range of approximately 1 to 7 Hz. The disk and ring ideally rotate at 800 RPM in one direction, then decelerate to a stop before re-accelerating back to 800 RPM in the opposite direction. During this process, the apparatus 300 is moved back and forth on the skin to accentuate the effects of the reversing abrasion and repeatedly expose all hair strands on the skin to the counter-rotating abrasive surfaces 328 and 334. This process is continued as the hair is knotted and flexed and broken off at the roots (for longer hairs), or worn away (for shorter hairs).

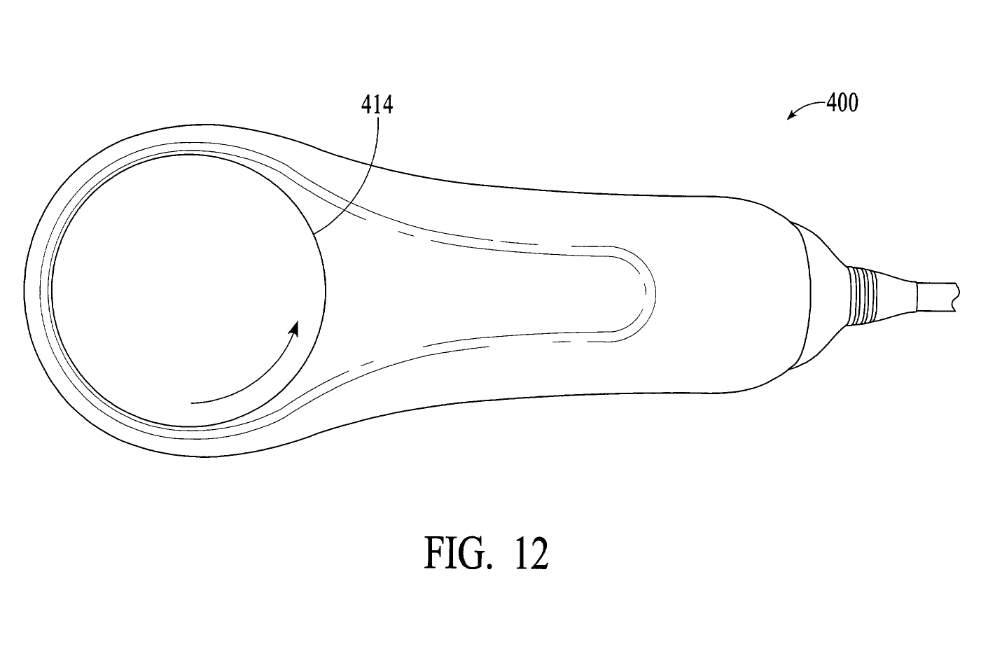

A fourth embodiment 400 is depicted in FIG. 12 wherein the circuitry functions similarly to the third embodiment, but only a single rotating disk 414 is used. The disk rotates in a first direction, say clockwise, then after one to three seconds or fourteen to forty-two complete rotations, the motor stops and reverses direction, and thus the disk's rotation direction is reversed, so that the disk rotates counter-clockwise. Again, after one to three seconds or fourteen to forty-two complete rotations in this direction, the motor direction is reversed so that the disk rotation reverses again.

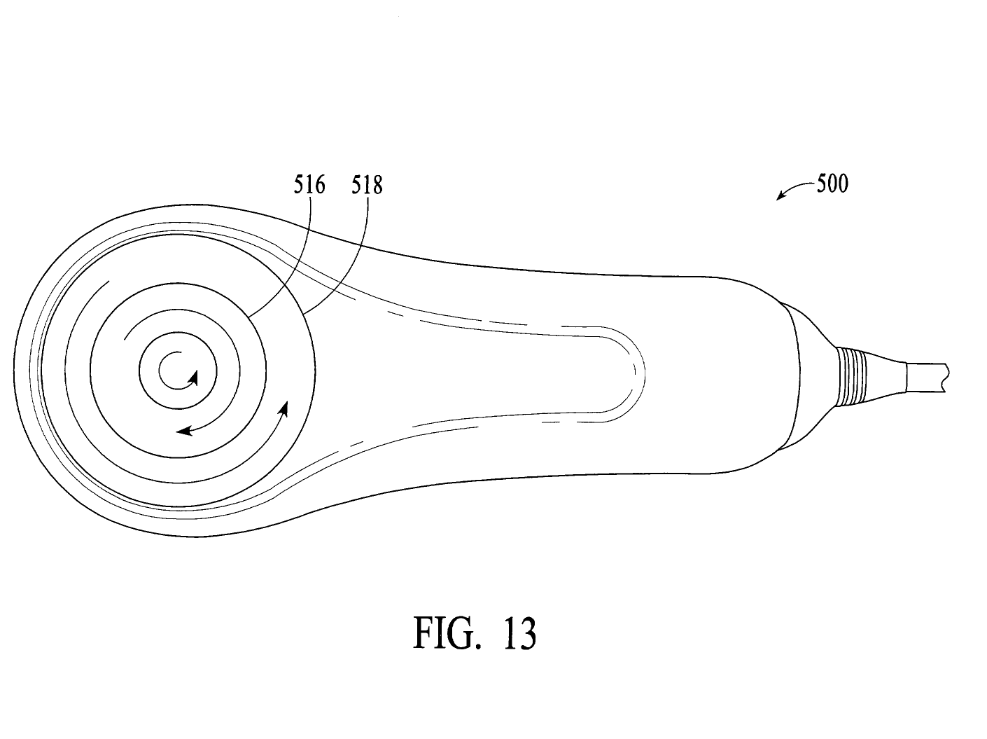

A fifth embodiment, 500, shown in FIG. 13, is similar to the preferred embodiment 100, except that it additionally comprises a second abrasive ring 518, concentric with and surrounding the first abrasive ring, 516, and arranged to rotate oppositely there-from. Use is otherwise identical to use of the preferred embodiment.

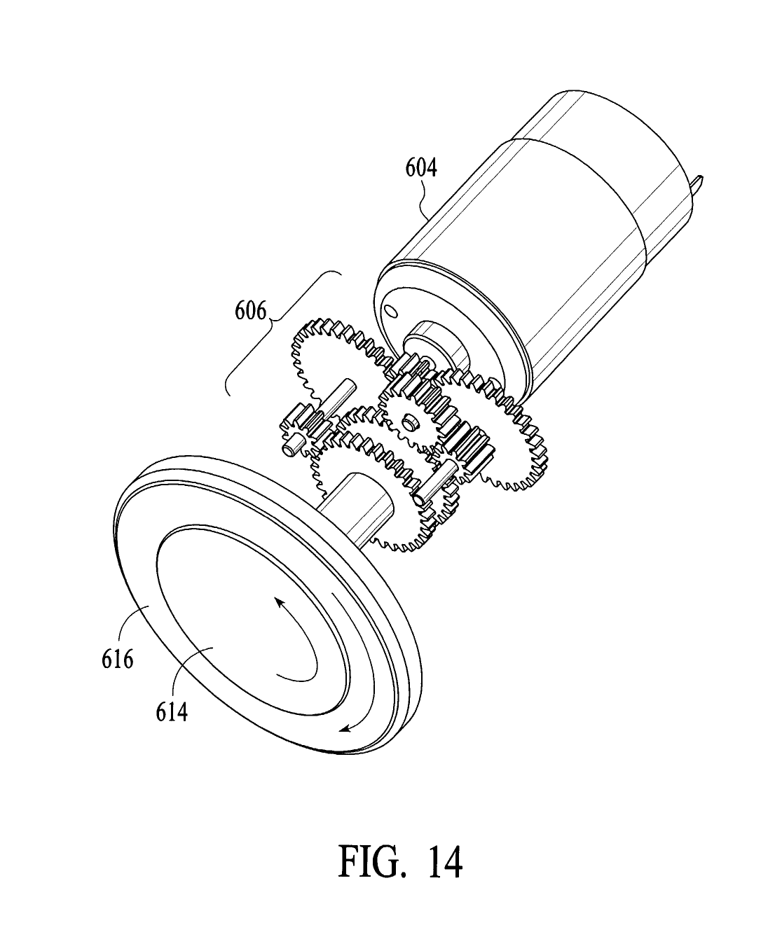

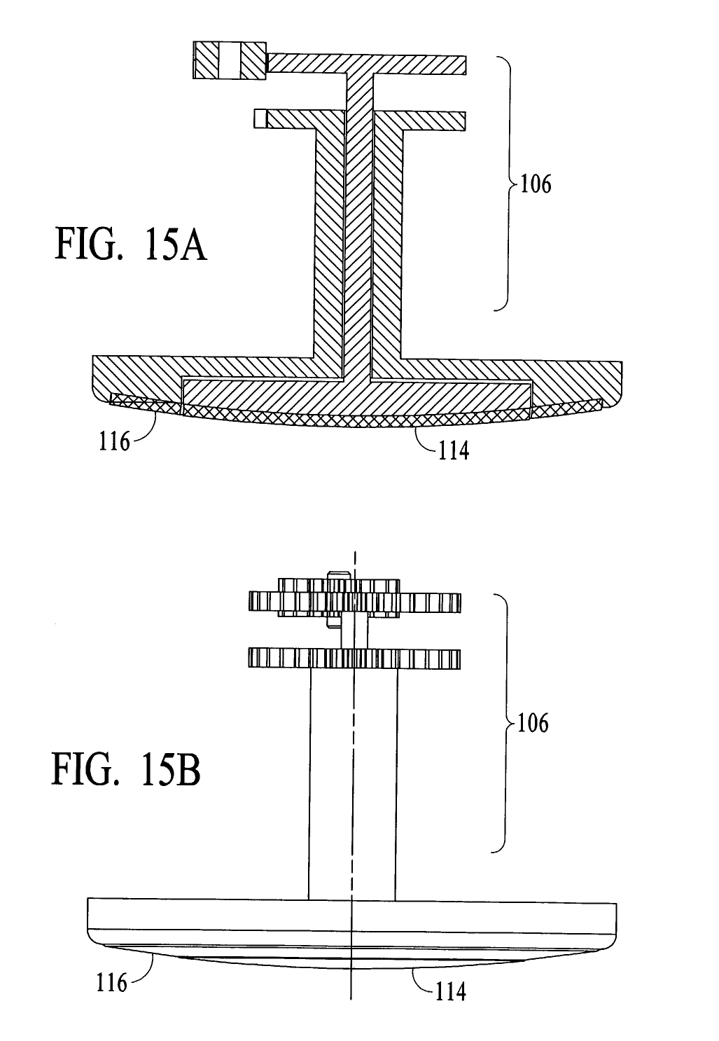

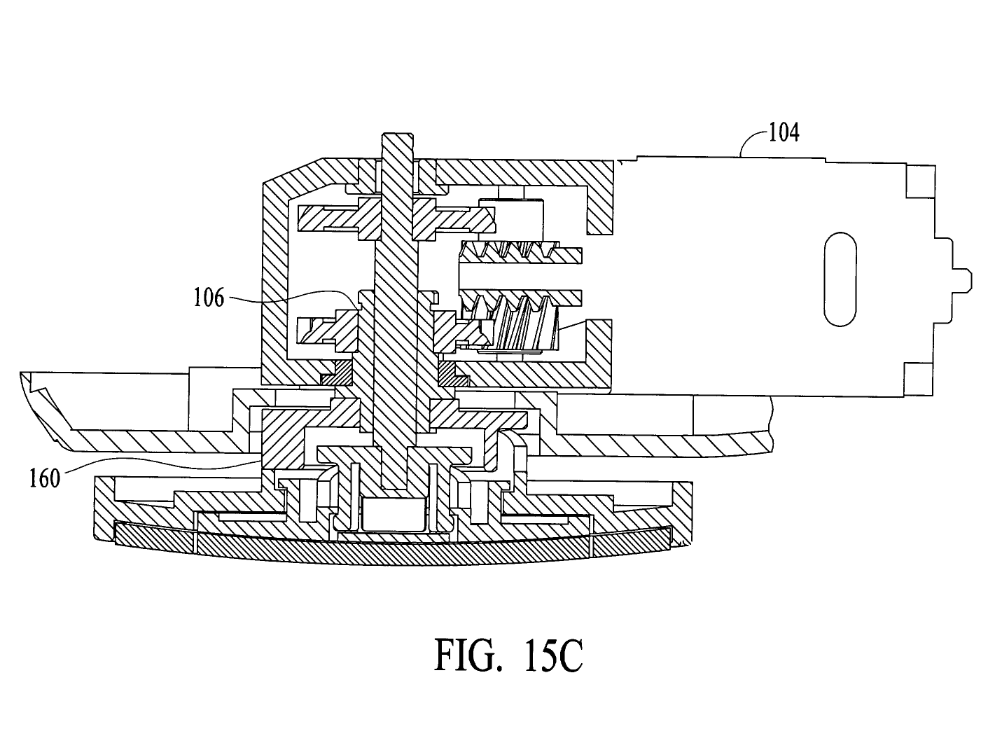

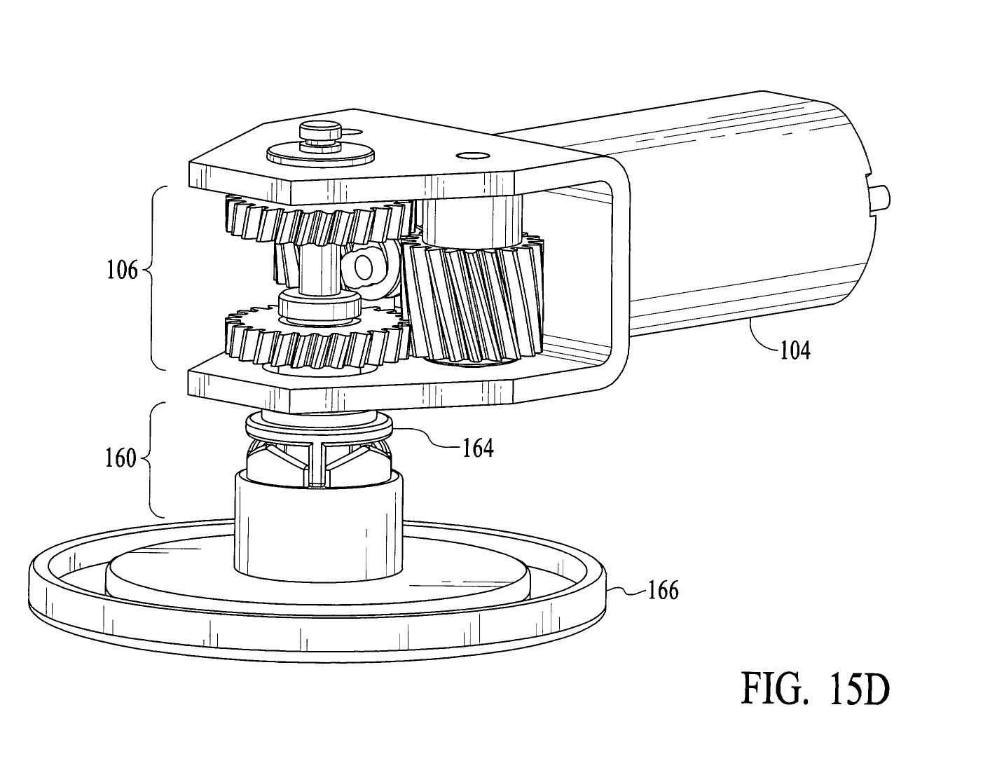

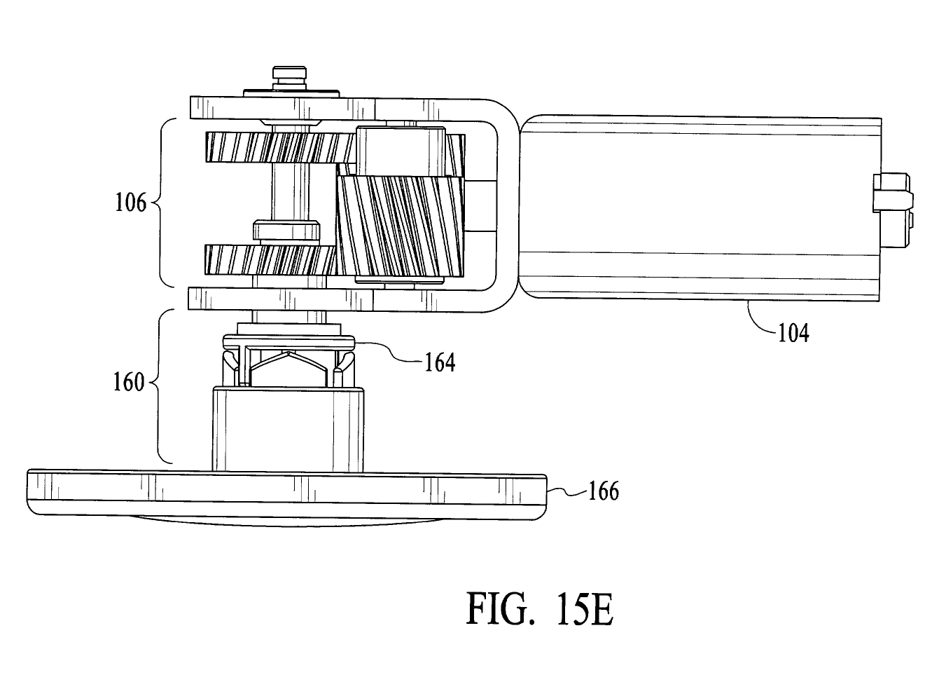

FIG. 14 depicts an alternate gear system 606 which may be employed in sixth embodiment, similar to the preferred embodiment 100 of aforementioned FIGS. 1 though 7 for use with motor 604 to drive a ring pad 616 and a disk pad 614 in opposite directions.

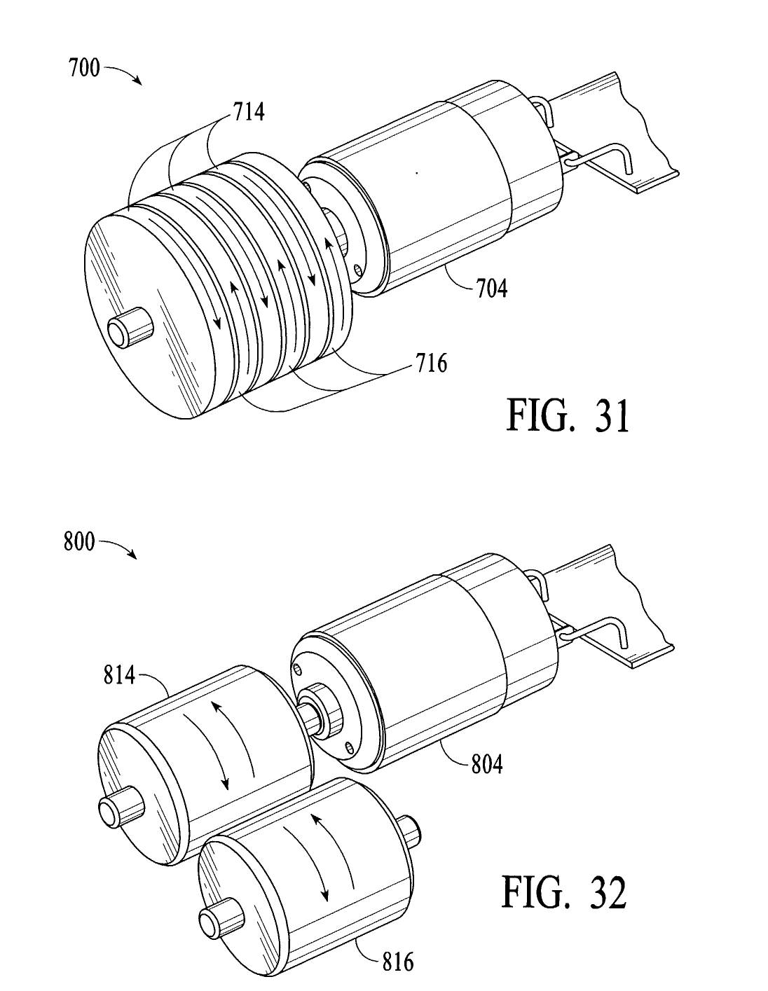

FIG. 31 depicts the drive system 700 of a seventh embodiment of the invention in which motor 704 causes a plurality of coaxial cylindrical abrasive surfaces 714 and 716 to rotate oppositely from each adjacent thereto.

FIG. 32 depicts the drive system 800 of an eighth embodiment of the invention in which motor 804 causes a pair of parallel cylindrical abrasive surfaces 814 and 816 to rotate oppositely from each other, either continuously in the same directions, or in cyclically reversing directions.

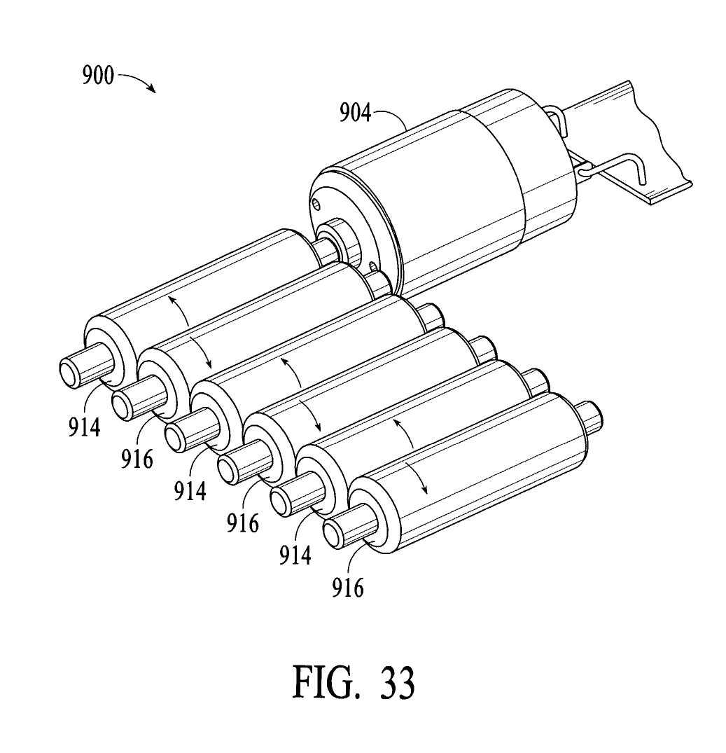

FIG. 33 depicts the drive system 900 of a ninth embodiment of the invention in which motor 904 causes a plurality of parallel cylindrical abrasive surfaces 914 and 916 to rotate oppositely from each adjacent thereto, either continuously in the same directions, or in cyclically reversing directions.

It should be understood that the invention herein embodied is to be taken as representative only and that various changes in shape, size, and arrangement of the parts may be made without departing from the spirit or scope of thereof The scope of the invention should only therefore be limited by the following claims.

I claim:

1. An electrical hair-removing device comprising an abrasive surface for pressing against hairy skin to exfoliate the skin and abrade the hair there-from, said surface adapted for automatic and continual electronically-controlled movement in differing directions relative the skin.

2. The hair-removing device of claim 1 wherein said movement is rotational and said differing directions are clockwise and counter-clockwise rotations.

3. An electrical hair-removing device comprising two or more adjacent and substantially coplanar abrasive surfaces for pressing against hairy skin to exfoliate the skin and abrade the hair there-from, each surface adapted for movement in differing directions relative to the other of said surfaces adjacent thereto.

4. The hair-removing device of claim 3 wherein said movement is rotational and said differing directions are clockwise and counterclockwise rotations.

5. The hair-removing device of claim 4 wherein said two or more abrasive surfaces comprise concentrically disposed surfaces rotating about a single rotational axis.

6. An electrical hair-removing device comprising a moving abrasive surface for pressing against hairy skin to exfoliate the skin and abrade the hair there-from, said surface adapted for alternating electronically-controlled movement in differing directions relative to the skin.

7. The hair-removing device of claim 6 wherein said movement is rotational and said differing directions are clockwise and counterclockwise rotations.

8. An electrical hair-removing device comprising two or more adjacent and substantially coplanar abrasive surfaces for pressing against hairy skin to exfoliate the skin and abrade the hair there-from, each surface adapted for alternating movement in differing directions relative to the other of said surfaces adjacent thereto.

9. The hair-removing device of claim 8 wherein said movement is rotational and said differing directions are clockwise and counterclockwise rotations.

10. The hair-removing device of claim 9 wherein said two or more abrasive surfaces comprise concentrically disposed surfaces rotating about a single rotational axis.

11. An electrical hair-removing device comprising a movable abrasive surface for pressing against hairy skin to exfoliate the skin and abrade the hair there-from, wherein said abrasive surface is removable from said device for replacement thereof and comprises a rotating abrasive inner disk concentrically surrounded by a counter-rotating abrasive outer ring.

12. The hair-removing device of claim 11 wherein said abrasive surface is spherical.

13. The hair-removing device of claim 11 wherein said abrasive surface is removable from said device by bayonet-type attachment means.

14. The hair-removing device of claim 11 wherein said abrasive surface is removable from said device by thread means.

15. The hair-removing device of claim 11 wherein said abrasive surface is removable from said device by hook and loop fastening means.

16. An abrasive element for use in a hair removing device and comprising an inner disk and a separate outer ring concentric therewith.

17. A method of removing hair from hairy skin and exfoliating the skin comprising;
providing an abrasive surface comprising an electrically rotated abrasive inner disk and an electrically counter-rotated abrasive outer ring,
pressing said abrasive surface against the skin until the hair is removed from the skin and the skin is exfoliated.

18. A method of removing hair from hairy skin and exfoliating the skin comprising,
Providing an abrasive surface comprising at least one electrically rotated abrasive first disk and at least one electrically rotated abrasive second disk adjacent to said first disk and adapted for opposite rotation relative thereto,
Pressing said abrasive surface against the skin until the hair is removed from the skin and the skin is exfoliated.

19. An electrical hair-removing device comprising two or more adjacent and substantially parallel abrasive cylinders for pressing against hairy skin to exfoliate the skin and abrade the hair there-from, each cylinder adapted for rotational movement in differing directions relative to the other of said cylinders adjacent thereto.

20. The hair-removing device of claim 19 wherein said differing directions are clockwise and counterclockwise rotations.

21. An electrical hair-removing device comprising two or more adjacent and coaxial abrasive cylinders for pressing against hairy skin to exfoliate the skin and abrade the hair there-from, each cylinder adapted for rotational movement in differing directions relative to the other of said cylinders adjacent thereto.

22. The hair-removing device of claim 21 wherein said differing directions are clockwise and counterclockwise rotations.

23. In an electrical hair removing device of the type having a stationary body and one or more rotating shafts extending there-from, and one or more abrasive surfaces adapted for connection to the one or more rotating shafts and adapted for rotation therewith relative to the housing, the improvement comprising;
One or more connection couplings for enabling connection and removal of said one or more abrasive surfaces to and from said one or more shafts, wherein said one or more connection couplings each comprise longitudinal locking means for fixed longitudinal coupling of said one or more abrasive surfaces to said one or more shafts, and rotational locking means for fixed rotational coupling of said one or more abrasive surfaces to said one or more shafts.

24. The improvement of claim 23 wherein each abrasive surface comprises an abrasive pad and each coupling comprises a pad connector fixed to said pad and a drive connector fixed to said shaft, and wherein each pad connector is adapted for connection to each drive connector by an axial connection force, and wherein each one of said pad connectors or said drive connectors contains one or more lead-in cams and rotational locking slots and each other of said pad connectors or said drive connectors contains one or more rotational locking tabs, and each cam is adapted to rotate each associated tab into rotational alignment with each associated slots by means of said axial connecting force.

25. The improvement of claim 23 wherein each abrasive surface comprises an abrasive pad and each coupling comprises a pad connector fixed to said pad and a drive connector fixed to said shaft, and wherein each pad connector is adapted for connection to each drive connector by an axial connection force, and wherein each one of said pad connectors contains one or more lead-in cams and rotational locking slots and each drive connector contains one or more rotational locking tabs, and each cam is adapted to rotate each associated tab into rotational alignment with each associated slots by means of said axial connecting force.
